(19) United States
(12) Patent Application Publication
(10) Pub. No.: US 2020/0054800 A1
(43) Pub. Date: Feb. 20, 2020

(54) Catheter and Method for Inducing Negative Pressure in a Patient's Bladder

(71) Applicant: Strataca Systems Limited, Floriana (MT)

(72) Inventors: Andrew Wilbourn, Crestwood, KY (US); Morgan Hinchey, Woodstock, GA (US); Colin Huber, Roswell, GA (US); Daisy Smith, Marietta, GA (US); John R. Erbey, II, Milton, GA (US); Jacob L. Upperco, Atlanta, GA (US); David E. Orr, Piedmont, SC (US)

(21) Appl. No.: 16/662,212

(22) Filed: Oct. 24, 2019

Related U.S. Application Data
(63) Continuation of application No. PCT/US2018/029310, filed on Apr. 25, 2018.
Application No. PCT/US2018/029310 is a continuation-in-part of application No. 15/879,770, filed on Jan. 25, 2018.
Application No. 15/879,770 is a continuation-in-part of application No. 15/687,064, filed on Aug. 25, 2017.
Application No. 15/687,064 is a continuation-in-part of application No. 15/411,884, filed on Jan. 20, 2017, now Pat. No. 10,512,713.
Application No. 15/411,884 is a continuation-in-part of application No. 15/214,955, filed on Jul. 20, 2016, now Pat. No. 10,307,564.
Application No. 15/879,770 is a continuation-in-part of application No. 15/687,083, filed on Aug. 25, 2017.
Application No. 15/687,083 is a continuation-in-part of application No. 15/411,884, filed on Jan. 20, 2017, now Pat. No. 10,512,713.
Application No. 15/411,884 is a continuation-in-part of application No. 15/214,955, filed on Jul. 20, 2016, now Pat. No. 10,307,564.
Application No. 15/879,770 is a continuation-in-part of application No. 15/745,823, filed on Jan. 18, 2018.
(60) Provisional application No. 62/300,025, filed on Feb. 25, 2016.
(60) Provisional application No. 62/278,721, filed on Jan. 14, 2016.
(60) Provisional application No. 62/260,966, filed on Nov. 30, 2015.
(60) Provisional application No. 62/194,585, filed on Jul. 20, 2015.
(60) Provisional application No. 62/300,025, filed on Feb. 25, 2016.
(60) Provisional application No. 62/278,721, filed on Jan. 14, 2016.
(60) Provisional application No. 62/260,966, filed on Nov. 30, 2015.
(60) Provisional application No. 62/194,585, filed on Jul. 20, 2015.
(60) Provisional application No. 62/300,025, filed on Feb. 25, 2016.
(60) Provisional application No. 62/278,721, filed on Jan. 14, 2016.
(60) Provisional application No. 62/260,966, filed on Nov. 30, 2015.
(60) Provisional application No. 62/194,585, filed on Jul. 20, 2015.
(60) Provisional application No. 62/489,789, filed on Apr. 25, 2017.
(60) Provisional application No. 62/489,831, filed on Apr. 25, 2017.

(51) Int. Cl. A61M 1/00; A61M 25/00; A61M 25/04

(52) U.S. Cl. CPC A61M 1/0023; A61M 1/008; A61M 25/0017; A61M 25/04; A61M 2230/207; A61M 2210/1085; A61M 2210/1089; A61M 2230/20; A61M 2205/0216

(57) ABSTRACT

A fluid collection catheter includes an elongated tube having a proximal portion, a distal portion having a distal end, and a sidewall extending between a proximal end and the distal end thereof defining at least one drainage lumen. The sidewall of the tube includes a drainage portion. The fluid collection catheter also includes a tissue support having at least a first flange including a central portion connected to the distal portion of the tube and an outer portion extending radially and axially therefrom. The first flange can be configured to be deployed in a patient's bladder to maintain the distal end of the tube at a position in the bladder. When deployed, the tissue support defines a three-dimensional shape of sufficient size to permit flow of fluid contained in the bladder from the bladder through the drainage portion of the tube to the at least one drainage lumen.

### CROSS-REFERENCE TO RELATED APPLICATIONS

This application is a continuation of PCT International Application No. PCT/US2018/029310, filed Apr. 25, 2018, which is a continuation-in-part of U.S. patent application Ser. No. 15/879,770, filed Jan. 25, 2018, which is a continuation-in-part of U.S. patent application Ser. No. 15/687,064, filed Aug. 25, 2017, which is a continuation-in-part of U.S. patent application Ser. No. 15/411,884 filed Jan. 20, 2017, which is a continuation-in-part of U.S. patent application Ser. No. 15/214,955 filed Jul. 20, 2016, which claims the benefit of U.S. Provisional Application No. 62/300,025 filed Feb. 25, 2016, U.S. Provisional Application No. 62/278,721, filed Jan. 14, 2016, U.S. Provisional Application No. 62/260,966 filed Nov. 30, 2015, and U.S. Provisional Application No. 62/194,585, filed Jul. 20, 2015, each of which is incorporated by reference herein in its entirety.

U.S. patent application Ser. No. 15/879,770 is also a continuation-in-part of U.S. patent application Ser. No. 15/687,083 filed Aug. 25, 2017, which is a continuation-in-part of U.S. patent application Ser. No. 15/411,884 filed Jan. 20, 2017, which is a continuation-in-part of U.S. patent application Ser. No. 15/214,955 filed Jul. 20, 2016, which claims the benefit of U.S. Provisional Application No. 62/300,025 filed Feb. 25, 2016, U.S. Provisional Application No. 62/278,721, filed Jan. 14, 2016, U.S. Provisional Application No. 62/260,966 filed Nov. 30, 2015, and U.S. Provisional Application No. 62/194,585, filed Jul. 20, 2015, each of which is incorporated by reference herein in its entirety.

U.S. patent application Ser. No. 15/879,770 is also a continuation-in-part of U.S. patent application Ser. No. 15/745,823 filed Jan. 18, 2018, which is the U.S. national phase of PCT International Application No. PCT/US2016/043101, filed Jul. 20, 2016, which claims the benefit of U.S. Provisional Application No. 62/300,025, filed Feb. 25, 2016, U.S. Provisional Application No. 62/278,721, filed Jan. 14, 2016, U.S. Provisional Application No. 62/260,966 filed Nov. 30, 2015, and U.S. Provisional Application No. 62/194,585, filed Jul. 20, 2015, each of which is incorporated by reference herein in its entirety.

U.S. patent application Ser. No. 15/879,770 claims the benefit of U.S. Provisional Application No. 62/489,789, filed Apr. 25, 2017, and U.S. Provisional Application No. 62/489,831, filed Apr. 25, 2017, each of which are incorporated by reference herein in its entirety.

### BACKGROUND

### Technical Field

The present disclosure relates to devices and methods for treating impaired renal function across a variety of disease states and, in particular, to devices and methods for collection of urine and inducement of negative and/or positive pressure in portions of a patient's urinary tract.

### Background

The renal or urinary system includes a pair of kidneys, each kidney being connected by a ureter to the bladder, and a urethra for draining urine produced by the kidneys from the bladder. The kidneys perform several vital functions for the human body including, for example, filtering the blood to eliminate waste in the form of urine. The kidneys also regulate electrolytes (e.g., sodium, potassium and calcium) and metabolites, blood volume, blood pressure, blood pH, fluid volume, production of red blood cells, and bone metabolism. Adequate understanding of the anatomy and physiology of the kidneys is useful for understanding the impact that altered hemodynamics other fluid overload conditions have on their function.

In normal anatomy, the two kidneys are located retroperitoneally in the abdominal cavity. The kidneys are bean-shaped encapsulated organs. Urine is formed by nephrons, the functional unit of the kidney, and then flows through a system of converging tubules called collecting ducts. The collecting ducts join together to form minor calyces, then major calyces, which ultimately join near the concave portion of the kidney (renal pelvis). A major function of the renal pelvis is to direct urine flow to the ureter. Urine flows from the renal pelvis into the ureter, a tube-like structure that carries the urine from the kidneys into the bladder. The outer layer of the kidney is called the cortex, and is a rigid fibrous encapsulation. The interior of the kidney is called the medulla. The medulla structures are arranged in pyramids.

Each kidney is made up of approximately one million nephrons. A schematic drawing of a nephron 1102 is shown in FIG. 25. Each nephron includes the glomerulus 1110, Bowman's capsule 1112, and tubules 1114. The tubules 1114 include the proximal convoluted tubule 1116, the loop of Henle 1118, the distal convoluted tubule 1120, and the collecting duct 1122. The nephrons 1102 contained in the cortex layer of the kidney are distinct from the anatomy of those contained in the medulla. The principal difference is the length of the loop of Henle 1118. Medullary nephrons contain a longer loop of Henle, which, under normal circumstances, allows greater regulation of water and sodium reabsorption than in the cortex nephrons.

The glomerulus is the beginning of the nephron, and is responsible for the initial filtration of blood. Afferent arterioles pass blood into the glomerular capillaries, where hydrostatic pressure pushes water and solutes into Bowman's capsule. Net filtration pressure is expressed as the hydrostatic pressure in the afferent arteriole minus the hydrostatic pressure in Bowman's space minus the osmotic pressure in the efferent arteriole.

Net Filtration Pressure=Hydrostatic Pressure (Afferent Arteriole)−Hydrostatic Pressure (Bowman's Space)−Osmotic Pressure (Efferent Arteriole) (Equation 1)

The magnitude of this net filtration pressure defined by Equation 1 determines how much ultra-filtrate is formed in Bowman's space and delivered to the tubules. The remaining blood exits the glomerulus via the efferent arteriole. Normal glomerular filtration, or delivery of ultra-filtrate into the tubules, is about 90 ml/min/1.73 m².

The glomerulus has a three-layer filtration structure, which includes the vascular endothelium, a glomerular basement membrane, and podocytes. Normally, large proteins such as albumin and red blood cells, are not filtered into Bowman's space. However, elevated glomerular pressures and mesangial expansion create surface area changes on the basement membrane and larger fenestrations between the podocytes allowing larger proteins to pass into Bowman's space.

Ultra-filtrate collected in Bowman's space is delivered first to the proximal convoluted tubule. Re-absorption and secretion of water and solutes in the tubules is performed by a mix of active transport channels and passive pressure gradients. The proximal convoluted tubules normally reabsorb a majority of the sodium chloride and water, and nearly all glucose and amino acids that were filtered by the glomerulus. The loop of Henle has two components that are designed to concentrate wastes in the urine. The descending limb is highly water permeable and reabsorbs most of the remaining water. The ascending limb reabsorbs 25% of the remaining sodium chloride, creating a concentrated urine, for example, in terms of urea and creatinine. The distal convoluted tubule normally reabsorbs a small proportion of sodium chloride, and the osmotic gradient creates conditions for the water to follow.

Under normal conditions, there is a net filtration of approximately 14 mmHg. The impact of venous congestion can be a significant decrease in net filtration, down to approximately 4 mmHg. See Jessup M., The cardiorenal syndrome: Do we need a change of strategy or a change of tactics?, JACC 53(7):597-600, 2009 (hereinafter “Jessup”). The second filtration stage occurs at the proximal tubules. Most of the secretion and absorption from urine occurs in tubules in the medullary nephrons. Active transport of sodium from the tubule into the interstitial space initiates this process. However, the hydrostatic forces dominate the net exchange of solutes and water. Under normal circumstances, it is believed that 75% of the sodium is reabsorbed back into lymphatic or venous circulation. However, because the kidney is encapsulated, it is sensitive to changes in hydrostatic pressures from both venous and lymphatic congestion. During venous congestion the retention of sodium and water can exceed 85%, further perpetuating the renal congestion. See Verbrugge et al., The kidney in congestive heart failure: Are natriuresis, sodium, and diruetucs really the good, the bad and the ugly? European Journal of Heart Failure 2014:16,133-42 (hereinafter “Verbrugge”).

Venous congestion can lead to a prerenal form of acute kidney injury (AKI). Prerenal AKI is due to a loss of perfusion (or loss of blood flow) through the kidney. Many clinicians focus on the lack of flow into the kidney due to shock. However, there is also evidence that a lack of blood flow out of the organ due to venous congestion can be a clinically important sustaining injury. See Damman K, Importance of venous congestion for worsening renal function in advanced decompensated heart failure, JACC 17:589-96, 2009 (hereinafter “Damman”).

Prerenal AKI occurs across a wide variety of diagnoses requiring critical care admissions. The most prominent admissions are for sepsis and Acute Decompensated Heart Failure (ADHF). Additional admissions include cardiovascular surgery, general surgery, cirrhosis, trauma, burns, and pancreatitis. While there is wide clinical variability in the presentation of these disease states, a common denominator is an elevated central venous pressure. In the case of ADHF, the elevated central venous pressure caused by heart failure leads to pulmonary edema, and, subsequently, dyspnea in turn precipitating the admission. In the case of sepsis, the elevated central venous pressure is largely a result of aggressive fluid resuscitation. Whether the primary insult was low perfusion due to hypovolemia or sodium and fluid retention, the sustaining injury is the venous congestion resulting in inadequate perfusion.

Hypertension is another widely recognized state that creates perturbations within the active and passive transport systems of the kidney(s). Hypertension directly impacts afferent arteriole pressure and results in a proportional increase in net filtration pressure within the glomerulus. The increased filtration fraction also elevates the peritubular capillary pressure, which stimulates sodium and water re-absorption. See Verbrugge.

Because the kidney is an encapsulated organ, it is sensitive to pressure changes in the medullary pyramids. The elevated renal venous pressure creates congestion that leads to a rise in the interstitial pressures. The elevated interstitial pressures exert forces upon both the glomerulus and tubules. See Verburgge. In the glomerulus, the elevated interstitial pressures directly oppose filtration. The increased pressures increase the interstitial fluid, thereby increasing the hydrostatic pressures in the interstitial fluid and peritubular capillaries in the medulla of the kidney. In both instances, hypoxia can ensue leading to cellular injury and further loss of perfusion. The net result is a further exacerbation of the sodium and water re-absorption creating a negative feedback. See Verbrugge, 133-42. Fluid overload, particularly in the abdominal cavity is associated with many diseases and conditions, including elevated intra-abdominal pressure, abdominal compartment syndrome, and acute renal failure. Fluid overload can be addressed through renal replacement therapy. See Peters, C. D., Short and Long-Term Effects of the Angiotensin II Receptor Blocker Irbesartanon Intradialytic Central Hemodynamics: A Randomized Double-Blind Placebo-Controlled One-Year Intervention Trial (the SAFIR Study), PLoS ONE (2015) 10(6): e0126882. doi:10.1371/journal.pone.0126882 (hereinafter “Peters”). However, such a clinical strategy provides no improvement in renal function for patients with the cardiorenal syndrome. See Bart B, Ultrafiltration in decompensated heart failure with cardiorenal syndrome, NEJM 2012;367:2296-2304 (hereinafter “Bart”).

In view of such problematic effects of fluid retention, devices and methods for improving removal of urine from the urinary tract and, specifically for increasing quantity and quality of urine output from the kidneys, are needed.

### SUMMARY

The present disclosure improves upon previous systems by providing a specialized (non-Foley) catheter for deployment within the bladder.

According to an aspect of the disclosure, a fluid collection catheter configured to be deployed in a bladder of a patient includes an elongated tube having a proximal portion configured for placement in a urethra of the patient, a distal portion having a distal end, and a sidewall extending between a proximal end and the distal end of the elongated tube defining at least one drainage lumen extending through the tube. The sidewall of the tube includes a drainage portion which allows fluid to pass through the sidewall and into the drainage lumen. The fluid collection catheter also includes a tissue support having at least a first flange including a central portion connected to the distal portion of the elongated tube and an outer portion extending radially and axially therefrom. The first flange can be configured to be deployed in the bladder to maintain the distal end of the elongated tube at a predetermined position in the bladder. When deployed, the tissue support defines a three-dimensional shape of sufficient size to permit flow of at least a portion of fluid contained in the bladder from the bladder through the drainage portion of the elongated tube to the at least one drainage lumen.

According to another aspect of the disclosure, a method of inducing a negative pressure to a urinary tract of a patient for enhancing urine excretion therefrom includes inserting a distal portion of an elongated tube of a urine collection catheter into the urinary tract. The elongated tube can include a proximal portion configured for placement in a urethra of the patient, a distal portion having a distal end, and a sidewall extending between a proximal end and the distal end of the elongated tube defining at least one drainage lumen extending through the tube. The sidewall of the elongated tube can include a drainage portion which allows fluid to pass through the sidewall and into the drainage lumen. The method further includes deploying a tissue support at a predetermined position in the patient's bladder. The tissue support can include at least one first flange having a central portion connected to the distal portion of the elongated tube and an outer portion extending axially and/or radially therefrom. The tissue support can be configured to be deployed in the bladder to maintain the distal end of the elongated tube at the predetermined position in the patient's bladder. When deployed, the tissue support defines a three-dimensional shape of sufficient size to permit flow of at least a portion of fluid contained in the bladder from the bladder through the drainage portion of the elongated tube to the at least one drainage lumen. The method also includes a step of inducing a negative pressure through the at least one drainage lumen of the elongated tube to draw at least a portion of fluid contained in the bladder from the bladder through the drainage portion of the elongated tube to the at least one drainage lumen.

According to another aspect of the disclosure, a system for drawing urine from a urinary tract of a patient includes a urine collection catheter configured to be deployed in a bladder of a patient and a pump in fluid connection with the catheter. The urine collection catheter can include an elongated tube including a proximal portion configured for placement in a urethra of the patient, a distal portion having a distal end, and a sidewall extending between a proximal end and the distal end of the elongated tube defining at least one drainage lumen extending through the tube. The sidewall of the tube can include a drainage portion which allows fluid to pass through the sidewall and into the drainage lumen. The urine collection catheter also includes a tissue support including at least a first flange having a central portion connected to the distal portion of the elongated tube and an outer portion extending radially and/or axially therefrom. The first flange can be configured to be deployed in the bladder to maintain the distal end of the elongated tube at a predetermined position in the bladder. When deployed, the tissue support defines a three-dimensional shape of sufficient size to permit flow of at least a portion of fluid contained in the bladder from the bladder through the drainage portion of the elongated tube to the at least one drainage lumen. The pump, which is in fluid connection with the drainage lumen of the elongated tube, is configured to introduce an internal negative pressure through the drainage lumen to the urinary tract of the patient to draw urine from the urinary tract.

Non-limiting examples of the present invention will now be described in the following numbered clauses:

Clause 1: A fluid collection catheter configured to be deployed in a bladder of a patient, comprising: an elongated tube comprising a proximal portion configured for placement in a urethra of the patient, a distal portion comprising a distal end, and a sidewall extending between a proximal end and the distal end of the elongated tube defining at least one drainage lumen extending through the tube, the sidewall comprising a drainage portion which allows fluid to pass through the sidewall and into the drainage lumen; and a tissue support comprising at least a first flange comprising a central portion connected to the distal portion of the elongated tube and an outer portion extending radially and axially therefrom, the first flange being configured to be deployed in the bladder to maintain the distal end of the elongated tube at a predetermined position in the bladder, wherein, when deployed, the tissue support defines a three-dimensional shape of sufficient size to permit flow of at least a portion of fluid contained in the bladder from the bladder through the drainage portion of the elongated tube to the at least one drainage lumen.

Clause 2: The catheter of clause 1, wherein the at least one flange is configured to transition from a retracted position in which at least a portion of a proximally facing surface of the first flange contacts an outer surface of the sidewall of the elongated tube, to a deployed position in which the portion of the proximally facing surface of the first flange is spaced apart from the sidewall.

Clause 3: The catheter of clause 1 or clause 2, wherein, when deployed in the patient's bladder, the tissue support is configured to maintain a volume of the three dimensional shape when an interior of the bladder is exposed to an internal negative pressure.

Clause 4: The catheter of any of clauses 1-3, wherein, when deployed in the patient's bladder, the tissue support is configured to maintain a volume of the three dimensional shape when an interior of the bladder is exposed to an internal negative pressure of from about 0.1 mmHg to about 150 mmHg.

Clause 5: The catheter of any of clauses 1-4, wherein, when deployed in the patient's bladder, the tissue support is configured to inhibit any portion of the bladder wall from occluding or obstructing ureteral orifices of the bladder upon delivery of negative pressure to the bladder through the drainage lumen of the tube.

Clause 6: The catheter of any of clauses 1-5, wherein the first flange has a maximum outer diameter of from about 10 mm to about 100 mm.

Clause 7: The catheter of any of clauses 1-6, wherein a height of the first flange is from about 10 mm to about 100 mm.

Clause 8: The catheter of any of clauses 1-7, wherein the elongated tube has an outer diameter of from about 0.5 mm to about 10 mm.

Clause 9: The catheter of any of clauses 1-8, wherein the elongated tube has an inner diameter of from about 0.5 mm to about 9 mm.

Clause 10: The catheter of any of clauses 1-9, wherein, when deployed in the patient's bladder, the three dimensional shape has a volume of from 0.1 cm³ to 500 cm³.

Clause 11: The catheter of any of clauses 1-10, wherein the drainage portion of the sidewall comprises a perforated section of tubing comprising at least one perforation permitting fluid to flow through the sidewall of the elongated tube into the at least one drainage lumen.

Clause 12: The catheter of clause 11, wherein the at least one perforation has one or more shapes, each shape being selected from at least one of a circular shape, an elliptical shape, a square shape, a regular polygonal shape, an irregular circular shape, an irregular polygonal shape, or combinations thereof.

Clause 13: The catheter of clause 11 or clause 12, wherein the at least one perforation has a diameter of about 0.05 mm to about 2.0 mm.

Clause 14: The catheter of any of clauses 11-13, wherein, when deployed in the patient's bladder, the tissue support is configured to inhibit any portion of a wall of the bladder from occluding the at least one perforation of the drainage portion upon delivery of negative pressure to an interior of the bladder through the drainage lumen of the elongated tube.

Clause 15: The catheter of any of clauses 1-14, wherein, when deployed in the patient's bladder, the first flange comprises a distally facing dome-shaped surface extending radially outwardly and proximally from the sidewall of the elongated tube.

Clause 16: The catheter of clause 15, wherein the first flange comprises at least one radial slit extending from an outer edge of the flange radially inwardly toward the central portion of the flange.

Clause 17: The catheter of clause 16, wherein the first flange comprises a plurality of radial slits which at least partially separate petal portions of the flange, and wherein a distance between adjacent petal portions increases as the flange transitions to the deployed position.

Clause 18: The catheter of any of clauses 1-17, wherein the first flange comprises at least one perforation extending between a proximal surface and a distal surface of the flange, and wherein the at least one perforation is positioned to permit negative pressure to pass through the flange to other portions of the bladder.

Clause 19: The catheter of any of clauses 1-17, wherein the at least one first flange comprises a medical grade elastomeric polymer material.

Clause 20: The catheter of clause 19, wherein the elastomeric polymer material comprises one or more of silicone, thermoplastic polyurethane, or a composites of a silicone or a polyurethane and a metallic component.

Clause 21: The catheter of any of clauses 1-20, wherein the at least one first flange comprises silicone having a shore hardness of between about Shore 20 A and Shore 100 A.

Clause 22: The catheter of any of clauses 1-21, wherein the central portion of the at least one first flange comprises a collar slidably connected to the sidewall of the elongated tube configured to slide along the sidewall of the tube to adjust a position of the at least one first flange.

Clause 23: The catheter of any of clauses 1-22, further comprising at least one second flange comprising a central opening connected to the sidewall of the elongated tube at a position proximal to the at least one first flange.

Clause 24: The catheter of clause 23, wherein, upon application of pressure to a proximally facing surface of the second flange, the second flange transitions to a concave configuration.

Clause 25: The catheter of clause 23, wherein, when deployed in the patient's bladder, a proximally facing surface of the second flange is configured to contact a portion of an inferior portion of the bladder wall surrounding a urethra opening into the bladder.

Clause 26: The catheter of any of clauses 23-25, wherein an outer diameter of the second flange is greater than an outer diameter of the first flange.

Clause 27: The catheter of any of clauses 23-26, wherein the distal portion of the elongated tube comprises a telescoping tube comprising an inner tube slidably received in an outer tube for adjusting a distance between the first flange and the second flange.

Clause 28: The catheter of clause 27, wherein the first flange is connected to the inner tube and the second flange is connected to the outer tube.

Clause 29: The catheter of any of clauses 23-28, further comprising at least one third flange comprising a central portion connected to the distal end of the elongated tube and an outer portion extending therefrom, the third flange being configured to support a superior portion of the bladder wall.

Clause 30: The catheter of clause 29, wherein an outer diameter of the third flange is less than an outer diameter of the first flange and the second flange.

Clause 31: The catheter of any of clauses 1-30, further comprising a delivery catheter comprising a proximal end configured to remain external to the body, a distal end for insertion into the bladder, a sidewall extending therebetween, and at least one lumen sized to receive the elongated tube and tissue support, wherein the delivery catheter is configured to maintain the tissue support in a retracted position, in which at least a portion of a lower surface of the flange contacts the sidewall of the elongated tube, during insertion of the tissue support to the bladder of the patient.

Clause 32: The catheter of clause 31, wherein the delivery catheter has an inner diameter of from about 1.0 mm to about 20 mm.

Clause 33: The catheter of clause 31, wherein the at least one flange is biased to a deployed position, such that when pushed from the distal end of the delivery catheter, the at least one flange adopts its deployed configuration.

Clause 34: A method of inducing a negative pressure to a urinary tract of a patient for enhancing urine excretion therefrom, the method comprising: inserting a distal portion of an elongated tube of a urine collection catheter into the urinary tract, the elongated tube comprising a proximal portion configured for placement in a urethra of the patient, a distal portion comprising a distal end, and a sidewall extending between a proximal end and the distal end of the elongated tube defining at least one drainage lumen extending through the tube, the sidewall comprising a drainage portion which allows fluid to pass through the sidewall and into the drainage lumen; deploying a tissue support at a predetermined position in the patient's bladder, the tissue support comprising at least one first flange comprising a central portion connected to the distal portion of the elongated tube and an outer portion extending axially and/or radially therefrom, wherein the tissue support is configured to be deployed in the bladder to maintain the distal end of the elongated tube at the predetermined position in the patient's bladder, and wherein, when deployed, the tissue support defines a three-dimensional shape of sufficient size to permit flow of at least a portion of fluid contained in the bladder from the bladder through the drainage portion of the elongated tube to the at least one drainage lumen; and inducing a negative pressure through the at least one drainage lumen of the elongated tube to draw at least a portion of fluid contained in the bladder from the bladder through the drainage portion of the elongated tube to the at least one drainage lumen.

Clause 35: The method of clause 34, wherein, when deployed in the patient's bladder, the tissue support is configured to maintain a volume of the three dimensional shape when an interior of the bladder is exposed to the negative pressure.

Clause 36: The method of clause 34, wherein, when deployed in the patient's bladder, the tissue support is configured to maintain a volume of the three dimensional shape when an interior of the bladder is exposed to an internal negative pressure of from about 0.1 mmHg to about 150 mmHg.

Clause 37: The method of any of clauses 34-36, wherein inducing the negative pressure in the drainage lumen comprises coupling a mechanical pump to the proximal end of the drainage lumen to draw urine from the bladder into the drainage lumen through the drainage portion of the sidewall.

Clause 38: The method of any of clauses 34-37, wherein inducing the negative pressure comprises applying a negative pressure of from about 0.1 mmHg to about 150 mmHg to the proximal end of the elongated tube.

Clause 39: The method of any of clauses 34-38, wherein the elongated tube is inserted into the bladder in a delivery catheter, and wherein deploying the tissue support comprises retracting the delivery catheter to expose the at least one flange.

Clause 40: The method of clause 39, wherein the at least one first flange is biased to adopt a deployed position when the delivery catheter is retracted.

Clause 41: The method of any of clauses 34-40, wherein the tissue support further comprises at least a second flange comprising a central opening connected to the sidewall of the elongated tube at a position proximal to the first flange.

Clause 42: The method of clause 41, wherein deploying the tissue support comprises adjusting a position of the second flange such that a proximally facing surface of the second flange contacts a portion of an inferior portion of the bladder wall surrounding a urethra opening into the bladder.

Clause 43: The method of clause 41 or clause 42, wherein the tissue support further comprises at least one third flange comprising a central portion connected to the distal end of the elongated tube and an outer portion extending therefrom, and wherein, when negative pressure is applied to the bladder, the third flange supports a superior portion of the bladder wall.

Clause 44: A system for drawing urine from a urinary tract of a patient, the system comprising: a urine collection catheter configured to be deployed in a bladder of a patient comprising: an elongated tube comprising a proximal portion configured for placement in a urethra of the patient, a distal portion comprising a distal end, and a sidewall extending between a proximal end and the distal end of the elongated tube defining at least one drainage lumen extending through the tube, the sidewall comprising a drainage portion which allows fluid to pass through the sidewall and into the drainage lumen; and a tissue support comprising at least a first flange comprising a central portion connected to the distal portion of the elongated tube and an outer portion extending radially and/or axially therefrom, the first flange being configured to be deployed in the bladder to maintain the distal end of the elongated tube at a predetermined position in the bladder, wherein, when deployed, the tissue support defines a three-dimensional shape of sufficient size to permit flow of at least a portion of fluid contained in the bladder from the bladder through the drainage portion of the elongated tube to the at least one drainage lumen; and a pump in fluid connection with the drainage lumen of the elongated tube, wherein the pump is configured to introduce an internal negative pressure through the drainage lumen to the urinary tract of the patient to draw urine from the urinary tract.

Clause 45: The system of clause 44, wherein, when deployed in the patient's bladder, the tissue support is configured to maintain a volume of the three dimensional shape when an interior of the bladder is exposed to the internal negative pressure.

Clause 46: The system of clause 44 or clause 45, wherein, when deployed in the patient's bladder, the permeable tissue support is configured to maintain a volume of the three dimensional shape when an interior of the bladder is exposed to an internal negative pressure of from about 0.1 mmHg to about 150 mmHg.

Clause 47: The system of any of clauses 44-46, wherein the pump provides a sensitivity of about 10 mmHg or less.

Clause 48: The system of any of clauses 44-47, wherein the pump is configured to provide a negative pressure of from about 0.1 mmHg to about 150 mmHg.

Clause 49: The system of any of clauses 44-48, wherein the pump is configured to provide intermittent negative pressure.

Clause 50: The system of any of clauses 44-49, wherein the pump is configured to alternate between providing negative pressure and providing positive pressure.

Clause 51: The system of any of clauses 44-49, wherein the pump is configured to alternate between providing negative pressure and equalizing pressure to atmosphere.

Clause 52: The system of any of clauses 44-51, wherein the tissue support further comprises at least one second flange comprising a central opening connected to the sidewall of the elongated tube at a position proximal to the first flange.

Clause 53: The system of clause 52, wherein, upon application of pressure to a proximally facing surface of the second flange, a surface of the second flange transitions to a concave configuration.

Clause 54: The system of clause 52 or clause 53, wherein the tissue support further comprises at least one third flange comprising a central portion connected to the distal end of the elongated tube and an outer portion extending radially and axially therefrom, and wherein, when negative pressure is applied to the bladder, the third flange supports a superior portion of the bladder wall.

### BRIEF DESCRIPTION OF THE DRAWINGS

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limit of the invention.

Further features and other examples and advantages will become apparent from the following detailed description made with reference to the drawings in which:

FIG. 1 is a schematic drawing of a urine collection catheter deployed in the bladder of a male patient according to an example of the disclosure;

FIG. 2 is a schematic drawing of a urine collection system including the urine collection catheter of FIG. 1 deployed in a bladder of a patient and a fluid pump for providing negative pressure to the urinary tract according to an example of the disclosure;

FIG. 3 is a perspective view of the urine collection catheter of FIG. 1 according to an example of the disclosure;

FIG. 4 is a front view of the urine collection catheter of FIG. 3;

FIG. 5 is a cross-section view of the urine collection catheter of FIG. 4, taken along line 5-5;

FIG. 6 is a cross-sectional view of the urine collection catheter of FIG. 3 retracted in a delivery cannula prior to deployment in a bladder according to an example of the disclosure;

FIG. 7 is a cross-sectional view of the urine collection catheter of FIG. 3 deployed from a delivery cannula according to an example of the disclosure;

FIG. 8 is a cross-sectional view of the urine collection catheter of FIG. 3 retracted in a delivery cannula after being deployed in a bladder according to an example of the disclosure;

FIG. 9 is a front view of another embodiment of a urine collection catheter according to an example of the disclosure;

FIG. 10 is a front view of another embodiment of a urine collection catheter according to an example of the disclosure;

FIG. 11 is a front view of another embodiment of a urine collection catheter according to an example of the disclosure;

FIG. 12 is a schematic drawing of a urine collection system including a urine collection catheter deployed in a bladder and ureteral stents according to an example of the present disclosure;

FIG. 13 is a schematic drawing of an exemplary ureteral stent as is known in the prior art;

FIG. 14 is a schematic drawing of another example of a urine collection system including a urine collection catheter deployed in the bladder and ureteral stents including a helical retention portion according to an example of the present disclosure;

FIG. 15A is a schematic drawing of a helical retention portion of a ureteral stent according to an aspect of the disclosure;

FIG. 15B is a schematic drawing of the helical retention portion of the ureteral stent of FIG. 14A;

FIG. 16 is a flow chart illustrating a process for applying negative pressure using a ureteral and/or bladder catheter or urine collection assembly according to an example of the present disclosure;

FIG. 17 is a schematic drawing of a system for inducing negative pressure to the urinary tract of a patient according to an example of the present disclosure;

FIG. 18A is a plan view of a pump for use with the system of FIG. 17 according to an example of the present disclosure;

FIG. 18B is a side elevation view of the pump of FIG. 18A;

FIG. 19 is a flow chart illustrating a process for reducing creatinine and/or protein levels of a patient according to an example of the disclosure;

FIG. 20 is a flow chart illustrating a process for treating a patient undergoing fluid resuscitation according to an example of the disclosure;

FIG. 21 is a schematic drawing of an experimental set-up for evaluating negative pressure therapy in a swine model;

FIG. 22 is a graph of creatinine clearance rates for tests conducted using the experimental set-up shown in FIG. 21;

FIG. 23A is a low magnification photomicrograph of kidney tissue from a congested kidney treated with negative pressure therapy;

FIG. 23B is a high magnification photomicrograph of the kidney tissue shown in FIG. 23A;

FIG. 23C is a low magnification photomicrograph of kidney tissue from a congested and untreated (e.g., control) kidney;

FIG. 23D is a high magnification photomicrograph of the kidney tissue shown in FIG. 23C;

FIG. 24 is a graph of serum albumin relative to baseline for tests conduct on swine using the experimental method described herein; and

FIG. 25 is a schematic drawing of a nephron and surrounding vasculature showing a position of the capillary bed and convoluted tubules.

### DETAILED DESCRIPTION OF THE INVENTION

As used herein, the singular form of “a”, “an”, and “the” include plural referents unless the context clearly states otherwise.

As used herein, the terms “right”, “left”, “top”, and derivatives thereof shall relate to the invention as it is oriented in the drawing figures. The term “proximal” refers to the portion of the catheter device that is manipulated or contacted by a user and/or to a portion of an indwelling catheter nearest to the urinary tract access site. The term “distal” refers to the opposite end of the catheter device that is configured to be inserted into a patient and/or to the portion of the device that is inserted farthest into the patient's urinary tract. However, it is to be understood that the invention can assume various alternative orientations and, accordingly, such terms are not to be considered as limiting. Also, it is to be understood that the invention can assume various alternative variations and stage sequences, except where expressly specified to the contrary. It is also to be understood that the specific devices and processes illustrated in the attached drawings, and described in the following specification, are examples. Hence, specific dimensions and other physical characteristics related to the embodiments disclosed herein are not to be considered as limiting.

For the purposes of this specification, unless otherwise indicated, all numbers expressing quantities of ingredients, reaction conditions, dimensions, physical characteristics, and so forth used in the specification and claims are to be understood as being modified in all instances by the term “about.” Unless indicated to the contrary, the numerical parameters set forth in the following specification and attached claims are approximations that can vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Also, it should be understood that any numerical range recited herein is intended to include all sub-ranges subsumed therein. For example, a range of “1 to 10” is intended to include any and all sub-ranges between and including the recited minimum value of 1 and the recited maximum value of 10, that is, all sub-ranges beginning with a minimum value equal to or greater than 1 and ending with a maximum value equal to or less than 10, and all sub-ranges in between, e.g., 1 to 6.3, or 5.5 to 10, or 2.7 to 6.1.

As used herein, the terms “communication” and “communicate” refer to the receipt or transfer of one or more signals, messages, commands, or other type of data. For one unit or component to be in communication with another unit or component means that the one unit or component is able to directly or indirectly receive data from and/or transmit data to the other unit or component. This can refer to a direct or indirect connection that can be wired and/or wireless in nature. Additionally, two units or components can be in communication with each other even though the data transmitted can be modified, processed, routed, and the like, between the first and second unit or component. For example, a first unit can be in communication with a second unit even though the first unit passively receives data, and does not actively transmit data to the second unit. As another example, a first unit can be in communication with a second unit if an intermediary unit processes data from one unit and transmits processed data to the second unit. It will be appreciated that numerous other arrangements are possible.

Fluid retention and venous congestion are central problems in the progression to advanced renal disease. Excess sodium ingestion coupled with relative decreases in excretion leads to isotonic volume expansion and secondary compartment involvement. In some examples, the present invention is generally directed to devices and methods for facilitating drainage of urine or waste from the bladder, ureter, and/or kidney(s) of a patient. In some examples, the present invention is generally directed to devices and methods for inducing a negative pressure in the bladder, ureter, and/or kidney(s) of a patient. While not intending to be bound by any theory, it is believed that applying a negative pressure to the bladder, ureter, and/or kidney(s) can offset the medullary nephron tubule re-absorption of sodium and water in some situations. Offsetting re-absorption of sodium and water can increase urine production, decrease total body sodium, and improve erythrocyte production. Since the intra-medullary pressures are driven by sodium and, therefore, volume overload, the targeted removal of excess sodium enables maintenance of volume loss. Removal of volume restores medullary hemostasis. Normal urine production is 1.48-1.96 L/day (or 1-1.4 ml/min).

Fluid retention and venous congestion are also central problems in the progression of prerenal Acute Kidney Injury (AKI). Specifically, AKI can be related to loss of perfusion or blood flow through the kidney(s). Accordingly, in some examples, the present invention facilitates improved renal hemodynamics and increases urine output for the purpose of relieving or reducing venous congestion. Further, it is anticipated that treatment and/or inhibition of AKI positively impacts and/or reduces the occurrence of other conditions, for example, reduction or inhibition of worsening renal function in patients with NYHA Class III and/or Class IV heart failure. Classification of different levels of heart failure are described in The Criteria Committee of the New York Heart Association, (1994), Nomenclature and Criteria for Diagnosis of Diseases of the Heart and Great Vessels, (9th ed.), Boston: Little, Brown & Co. pp. 253-256, the disclosure of which is incorporated by reference herein in its entirety. Reduction or inhibition of episodes of AKI and/or chronically decreased perfusion may also be a treatment for Stage 4 and/or Stage 5 chronic kidney disease. Chronic kidney disease progression is described in National Kidney Foundation, K/DOQI Clinical Practice Guidelines for Chronic Kidney Disease: Evaluation, Classification and Stratification. Am. J. Kidney Dis. 39:S1-S266, 2002 (Suppl. 1), the disclosure of which is incorporated by reference herein in its entirety.

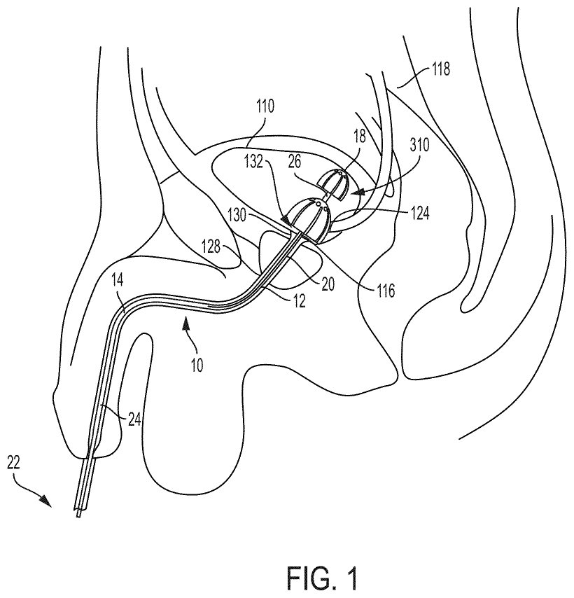

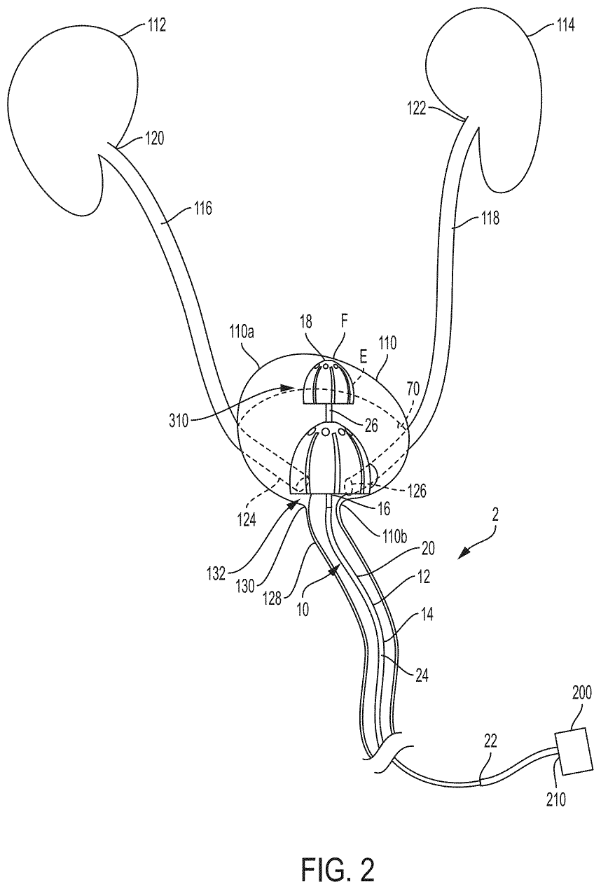

With reference to FIGS. 1 and 2, an exemplary system 2 for inducing negative pressure in a urinary tract of a patient for increasing renal perfusion is illustrated. It is noted, however, that the system 2 described herein is but one example of a negative pressure system for inducing negative pressure that can be used with urine collection catheters 10 disclosed herein. The catheters 10 and other elements disclosed herein can also be used with other medical devices for collecting fluid and/or providing negative pressure therapy within the scope of the present disclosure. In addition, in other examples, the urine collection catheters 10 disclosed herein can be connected to an unpressurized fluid collection container.

The system 2 comprises the urine collection catheter 10 and a pump 200 (shown in FIG. 2) for inducing the negative pressure in the urinary tract through the catheter 10. The patient's urinary tract comprises a patient's right kidney 112 and a left kidney 114. The kidneys 112, 114 are responsible for blood filtration and clearance of waste compounds from the body through urine. Urine produced by the right kidney 112 and the left kidney 114 is drained into a patient's bladder 110 through tubules, namely a right ureter 116 and a left ureter 118, which are connected to the kidneys 112, 114 at a renal pelvis 120, 122. Urine may be conducted through the ureters 116, 118 by peristalsis of the ureter walls, as well as by gravity. The ureters 116, 118 enter the bladder 110 through ureter orifices or openings 124, 126. As shown in FIG. 2, the ureteral orifices or openings 124, 126 are positioned at a midline of the bladder 110, approximately half way between an inferior bladder wall 110b and a superior bladder wall 110a (shown in FIG. 2). As such, proximal portions of the ureters 116, 118 are shown in phantom in FIG. 2 to indicate that such structures pass behind the bladder 110 and connect to the bladder 110 at the orifices or openings 124, 126.

The ureter orifices or openings 124, 126 are covered by soft tissue which essentially forms a one-way flap valve. When the bladder 110 is collecting urine, the soft tissue is able to accommodate pressure from the peristalsis, so that urine can pass from the ureters 116, 118 into the bladder 110. When the bladder 110 contracts to expel urine therefrom, the soft tissue is restrained against the ureter openings 124, 126 to prevent backflow of urine from the bladder 110 back into the ureters 116, 118. As described herein, in some examples, restraints, such as stents, catheters, tubes, and similar structures, can be positioned to allow the ureter openings 124, 126 to remain open during negative pressure therapy, so that the negative pressure can draw urine into the bladder 110 and into catheter devices positioned in the bladder 110.

The bladder 110 is a flexible and substantially hollow structure adapted to collect urine until the urine is excreted from the body. The bladder 110 is transitionable from an empty position (signified by reference line E in FIG. 2) to a full position (signified by reference line F in FIG. 2). Normally, when the bladder 110 reaches a substantially full state, urine is permitted to drain from the bladder 110 to a urethra 128 through a urethral sphincter or opening 130 located at a lower portion of the bladder 110. Contraction of the bladder 110 can be responsive to stresses and pressure exerted on a trigone region 132 (shown in FIG. 2) of the bladder 110, which is the triangular region extending between the ureteral openings 124, 126 and the urethral opening 130. The trigone region 132 is sensitive to stress and pressure, such that as the bladder 110 begins to fill, pressure on the trigone region 132 increases. When a threshold pressure on the trigone region 132 is exceeded, the ureteral sphincter or opening 130 relaxes and allows the bladder 110 to contract to expel collected urine through the urethra 128.

### Example Catheter Devices

With continued reference to FIGS. 1 and 2, the urine collection catheter 10 comprises an elongated tube 12 comprising a proximal portion 14, which can be configured for placement in the urethra 128 of the patient, a distal portion 16 comprising a distal end 18, and a sidewall 20 extending between a proximal end 22 and the distal end 18 of the elongated tube 12 defining at least one drainage lumen 24 extending through the tube 12. The sidewall 20 of the elongated tube 12 comprises a drainage portion 26, which allows fluid to pass through the sidewall 20 and into the drainage lumen 24.

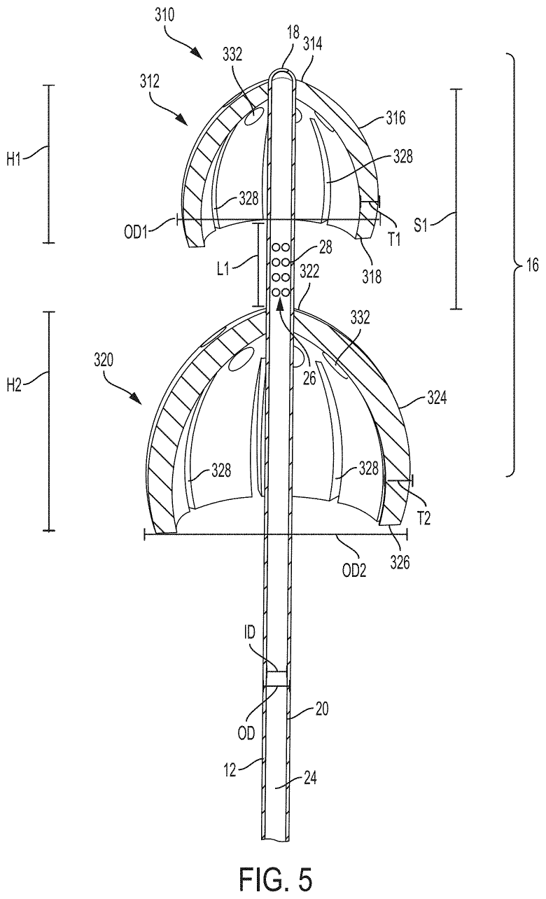

The elongated tube 12 can have any suitable length to accommodate anatomical differences for gender and/or patient size. In some examples, the tube 12 has a length from about 30 cm to about 120 cm. Further, the elongated tube 12 can have a maximum outer diameter OD (shown in FIG. 5) of about 0.25 mm to about 10 mm or, preferably, about 0.33 mm to about 3.0 mm. The elongated tube 12 can also have an inner diameter ID (shown in FIG. 5) of about 0.1 mm to 9.0 mm or, preferably, about 0.16 mm to about 2.40 mm. It is appreciated that the outer and inner diameters of the elongated tube 12 can include any of the subranges of the previously described ranges.

The elongated tube 12 can be formed from any suitable flexible and/or deformable material. Such materials facilitate advancing and/or positioning the elongated tube 12 in the bladder 110. Non-limiting examples of such materials include biocompatible polymers, polyvinyl chloride, polytetrafluoroethylene (PTFE) such as Teflon®, silicon coated latex, or silicon. At least a portion or all of the catheter 10, particularly the tube 12, can be coated with a hydrophilic coating to facilitate insertion and/or removal and/or to enhance comfort. In some examples, the coating is a hydrophobic and/or lubricious coating. For example, suitable coatings can comprise ComfortCoat® hydrophilic coating, which is available from Koninklijke DSM N.V. or hydrophilic coatings comprising polyelectrolyte(s) such as are disclosed in U.S. Pat. No. 8,512,795, which is incorporated herein by reference. In some examples, the tube 12 is impregnated with or formed from a material viewable by fluoroscopic imaging. For example, the biocompatible polymer which forms the tube 12 can be impregnated with barium sulfate or a similar radiopaque material. As such, the structure and position of the tube 12 is visible to fluoroscopy.

With specific reference to FIG. 2, aspects of the proximal portion 14 of the elongated tube 12 and external elements of the system 2 are described. The proximal portion 14 of the tube 12 is configured for placement in a portion of the urinary tract proximal to the bladder, such as the urethra 128. Proximal portions of the elongated tube 12 can also extend from the body and, for example, can be connected to a fluid collection container or to the pump 200. In some examples, the proximal portion 14 of the tube 12 is essentially free of or free of openings or perforations. While not intending to be bound by any theory, it is believed that when negative pressure is applied at the proximal portion 14 of the tube 12, that openings in the proximal portion of the tube 12 may be undesirable as such openings may diminish the negative pressure at the distal portion 16 of the urine collection catheter 10 and thereby diminish the draw or flow of fluid or urine from the kidney 112, 114, and renal pelvis 120, 122. It is desirable that the flow of fluid from the ureter 116, 118 and/or kidney 112, 114 is not prevented by occlusion of the ureter 116, 118 and/or kidney 112, 114 by the catheter 10.

In some examples, the proximal end 22 of the tube 12 comprises and/or is connected to a port 210 (shown in FIG. 2) for attaching the catheter 10 to the pump 200. The connection between the tube 12 and the pump 200 or another fluid collection container can be a standard connection mechanism, such as a luer lock or snap fit connection. In other examples, a dedicated or customized connector or connection device can be used for connecting the proximal end of the catheter device 10 or port 210 to other elements of the fluid collection system. In some examples, the customized connector can be structured to prevent a user from connecting the catheter 10 to unsuitable pressure sources. For example, the customized connector may be sized to prevent a user from connecting the catheter 10 to sources of wall suction or other sources of elevated vacuum pressures.

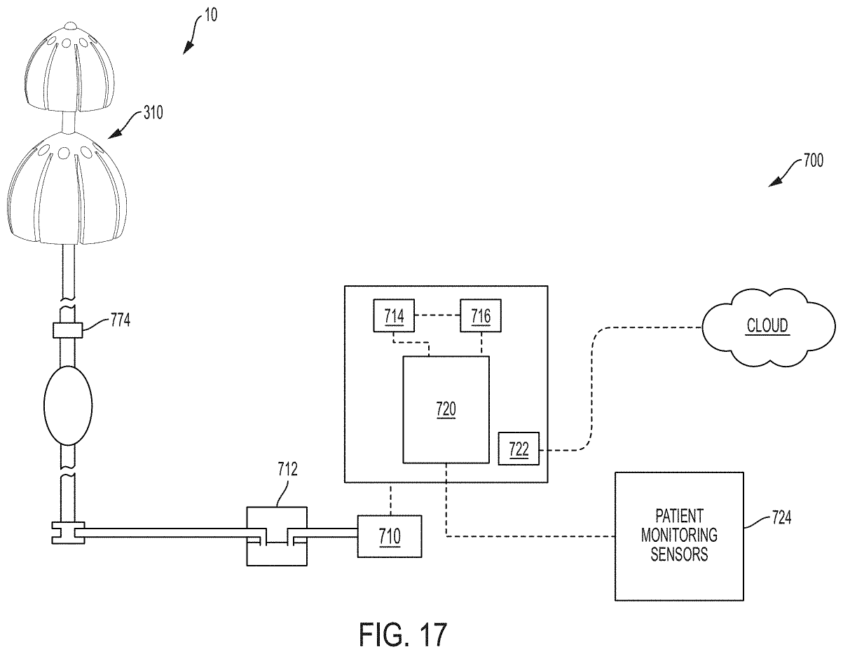

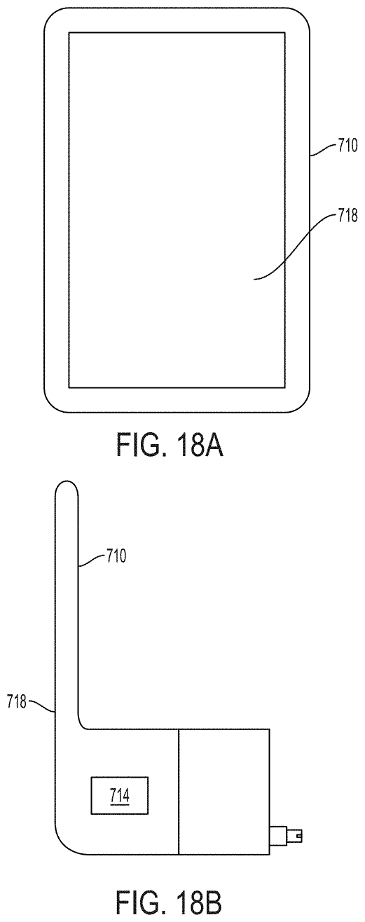

As described in further detail in connection with FIGS. 17-18B, the fluid pump 200 (shown in FIG. 2) is configured to generate a negative pressure to extract urine from the patient's urinary tract. In some examples, the pump 200 may also generate a positive pressure and, for example, may be configured to alternate between providing negative pressure, positive pressure, and equalizing pressure to atmosphere based on a selection from a user or automatically according to a predetermined schedule. The pump 200 can be configured to provide a low level negative pressure of from 0.10 mmHg to 150 mmHg to a proximal end of the catheter 10. In some examples, the pump 200 can be configured to operate at a number of discrete pressure levels. For example, the pump 200 may be configured to operate at predetermined pressure levels of 15 mmHg, 30 mmHg, and 45 mmHg. A user can select one of the pressure levels using a switch, dial, or controller as are known in the art.

A commercially available pump which can be adapted for use with the catheter 10 is the Air Cadet Vacuum Pump from Cole-Partner Instrument Company (Model No. EW-07530-85). The pump 200 can be connected in series to the regulator, such as the V-800 Series Miniature Precision Vacuum Regulator—1/8 NPT Ports (Model No. V-800-10-W/K), manufactured by Airtrol Components Inc. Pumps which can be adapted for use with the catheter 10 are also available from Ding Hwa Co., Ltd (DHCL Group) of Dacun, Changhua, China.

In other non-limiting examples, at least a portion of the pump 200 can be positioned within the patient's urinary tract, for example, within the bladder 110. For example, the pump 200 can comprise a pump module and a control module coupled to the pump module, the control module being configured to direct motion of the pump module. At least one of the pump module, the control module, or the power supply may be positioned within the patient's urinary tract. The pump module can comprise at least one pump element positioned within the fluid flow channel to draw fluid through the channel. Some examples of suitable pump assemblies, systems, and methods of use are disclosed in U.S. Patent Application No. 62/550,259 entitled “Indwelling Pump for Facilitating Removal of Urine from the Urinary Tract”, which is incorporated by reference herein in its entirety.

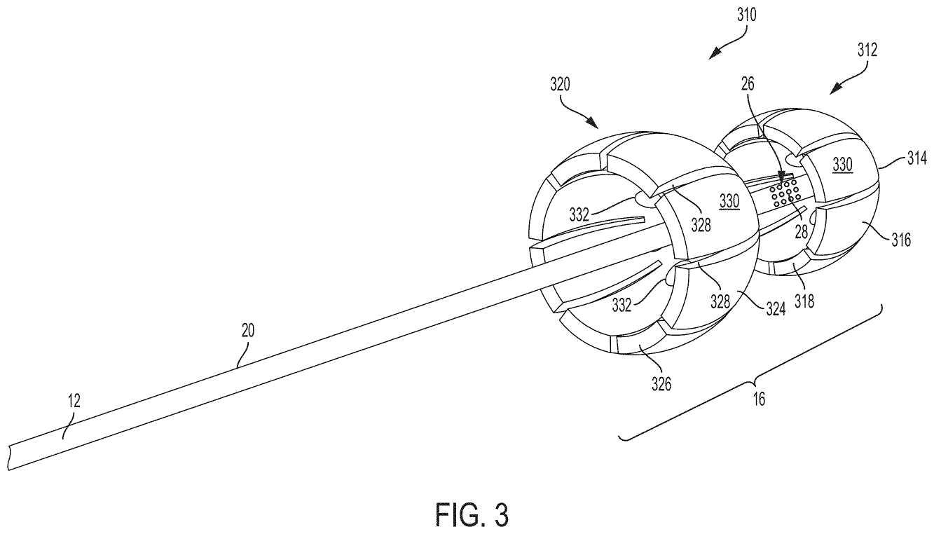

The drainage portion 26 of the drainage tube 12 can be provided in a variety of configurations depending on the fluid volume and flow rate intended to be drawn into the drainage lumen 24 from the bladder. For example, as shown in FIGS. 3-5, the drainage portion 26 comprises at least one opening or perforation 28 for permitting fluid to flow through the sidewall 20 of the tube 12 into the at least one drainage lumen 24. The openings or perforations 28 can have any shape desired, such as a circular shape, an elliptical shape, a square shape, a regular polygonal shape, an irregular circular shape, an irregular polygonal shape, or combinations thereof. In other examples, the drainage portion 26 of the elongated tube 12 comprises a perforated portion of sidewall 20 comprising a plurality of perforations.

Desirably, the perforations or fluid openings 28 are positioned so that negative pressure provided to the bladder 110 through the drainage lumen 24 is evenly distributed through the bladder 110. In some examples, the perforations or openings 28 are positioned so that negative pressure is provided from the drainage lumen 24 of the elongated tube 12 in all directions (e.g., so that a 360 degree negative pressure is provided to the bladder 110). In some examples, a diameter of the openings 28 can range from about 0.005 mm to about 1.0 mm. The configuration of each perforation or opening 28 can be the same or different, as desired. The perforations or openings 28 can be spaced in any arrangement, for example, linear or offset. In some examples, each perforation or opening 28 can be circular. In other examples, perforations 28 are non-circular. In other examples, the drainage portion 26 comprises a mesh material, for example, formed from a woven filament and comprising a plurality of openings for conducting fluid from the bladder 110 into the drainage lumen 24 of the tube 12.

Tissue Support with Two Flanges

Having described elements of the urine collection catheter 10 and pump 200, various structures 310, 410, 510, 610 for maintaining the distal portion 16 and distal end 18 of the elongated tube 12 at a desired position in the urinary tract, such as within the bladder, will now be discussed in detail. The tissue supports 310, 410, 510, 610 comprise one or more flanges connected to and extending radially and axially from an outer surface of the sidewall of the elongated tube 12. For example, the flanges can be convex or dome shaped structures extending radially and axially from the elongated tube 12. The one or more flanges can be formed from a flexible material so that the flanges can be retracted into a delivery cannula to remove the tissue support 310, 410, 510, 610 from the patient's bladder. The flanges should also be sufficiently strong to prevent the bladder from collapsing when negative pressure is applied thereto.

The flanges of the tissue supports 310, 410, 510, 610 can have a variety of sizes and configurations. With specific reference to FIGS. 3-5, an exemplary tissue support 310 for a bladder catheter 10 comprises at least a first or distal-most flange 312 comprising a central portion 314 connected to the distal portion 16 and/or the distal end 18 of the elongated tube 12 and an outer portion 316 extending radially and axially from the central portion 314 to an outer edge or rim 318. The tissue support 310 further comprises a proximal or second flange 320 comprising a central portion 322, outer portion 324, and outer rim 326. In some examples, the central portion 314 comprises an annular opening sized to receive the elongated tube 12, such that the annular opening engages a portion of the sidewall 20, thereby mounting the flange 312 to the tube 12. In that case, the distal end 18 of the tube 12 may extend through the flange 312. In that case, at least the distal portion 16 of the tube 12 can be formed from a flexible and/or compliant material to ensure that the distal end 18 of the tube 12 does not irritate or injure tissues of the bladder wall. In other examples, the central portion 314 can be a portion of the flange 312 that covers the distal end 18 of the elongated tube 12 to protect portions of the bladder wall from being irritated, abraded, or injured by the distal end 18 of the tube 12.

The flanges 312, 320 are configured to be deployed in the bladder to maintain the distal end 18 of the elongated tube 12 at a predetermined position in the bladder. For example, the flanges 312, 320 can be sized to define a three-dimensional volume within the bladder sufficient to prevent the ureteral orifices from occluding when the bladder collapses and/or when negative pressure is applied to the bladder. The three-dimensional shape refers to a regular shape defined by surfaces of the first flange 312 and/or the second flange 320. For example, for a catheter 10 including only a single flange, the three-dimensional shape is generally a semi-spherical shape defined by an outer surface of the flange and the outer rim 318 of the flange. For a catheter 10 including two or more flanges (as shown in FIGS. 3-5), the three dimensional shape is an elongated shape extending from an outer surface of a distal-most flange to the outer rim of the proximal most flange and sized to enclose the two of more flanges 312, 320. When negative pressure is applied to the bladder through a lumen 24 of the elongated tube 12, portions of the bladder wall are drawn against the flanges 312, 320. The flanges 312, 320 are positioned to counteract forces of the collapsing bladder, such that the bladder adopts the three-dimensional shape defined by the flange(s) 312, 320.

With continued reference to FIGS. 3-5, a distance S1 along the tube 12 between a proximally facing surface of the first flanges 312 and a distally facing surface of the second flange 320 can be selected based on the size of the trigone region of the bladder. For example, the flanges 312, 320 may be sized to span the trigone region, such that when deployed in the bladder, the first flange 312 extends above the trigone region and/or ureteral openings to prevent occlusion of the ureteral openings, and the second flange 320 sits below the trigone region and ureteral openings. For example, the distance S1 can be between about 5 mm to 30 mm (0.2 inch and 1.2 inches) and, preferably, about 8 mm (0.3 inch). An overall height H1 of the tissue support 310 (e.g., a distance along the tube 12 between the outer rim 326 of the second flange 320 and the distally facing surface of the first flange 312) is from about 40 mm to about 90 mm (1.5 inches and 3.5 inches), preferably, about 60 mm (2.4 inches).

As shown in FIGS. 3-5, in some examples, the drainage portion 26, which comprises the perforation or opening 28 in the sidewall 20 of the tube 12, is positioned between the first flange 312 and the second flange 320. In some instances, other portions of the elongated tube 12 are free from openings or perforations. When positioned between the first flange 312 and the second flange 320, the drainage portion 26 can have a longitudinal length L1 from about 5 mm to about 20 mm (0.2 inch to 0.8 inch).

In some examples, the flanges 312, 320 are formed from a compliant flexible material, which does not appreciably abrade, irritate, or damage a mucosal lining of the bladder walls or of a urethra when positioned adjacent to the mucosal lining of the bladder walls or the urethra. For example, the flanges 312, 320 can comprise a medical-grade elastomeric polymer material, such as silicone. In some examples, the flanges 312, 320 are formed from silicone having a Shore hardness of between Shore 20 A and Shore 100 A. Generally, a more flexible polymer material (e.g., a polymer having a shore hardness of around Shore 20 A to Shore 30 A) can be used when the flange 312, 320 is supported by a rigid frame, such as a frame comprising Nitinol. Other thermoplastic elastomers having sufficient flexibility and strength for supporting the bladder wall from collapsing can also be used. In other examples, the flanges 312, 320 can be formed from a composite of a polymer (e.g., a thermoplastic elastomer) and a metallic component, such as Nitinol.

In some examples, the second flange 320 is configured to contact a portion of the inferior bladder wall surrounding or adjacent to the urethral sphincter. In order to provide a stable anchor within the bladder, in this position, the second flange 320 may comprise a harder or more rigid material than the first flange 312. The first flange 312 is configured to provide support for the pliable and compliant bladder wall. For example, the first flange 312 may support portions of the bladder wall that collapse inwardly from both vertical and horizontal directions. As such, the first flange 312 can be formed from a softer, more compliant material. For example, the second flange 320 may have a durometer of about Shore 30 A, while the first flange 312 may have a durometer closer to Shore 60 A.

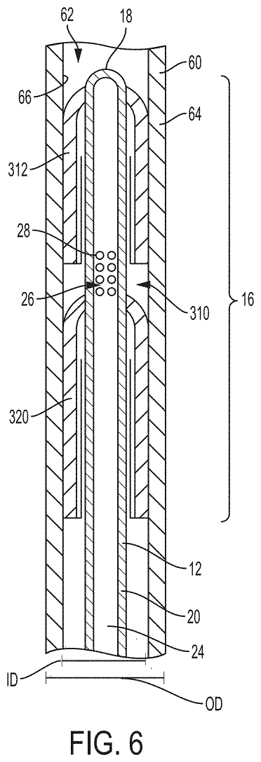

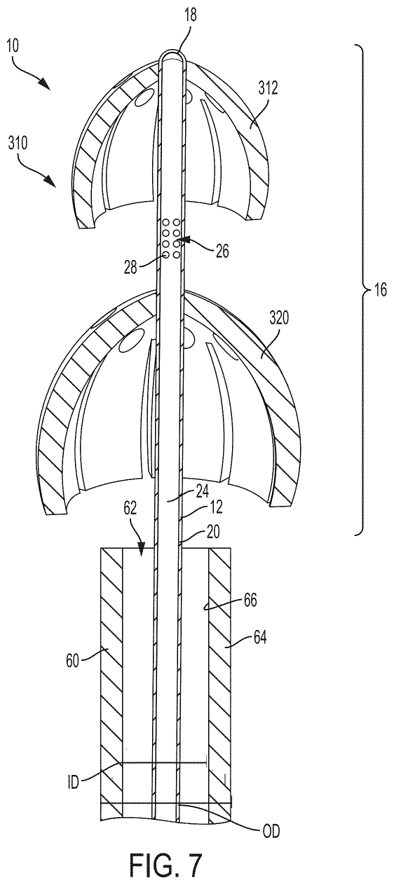

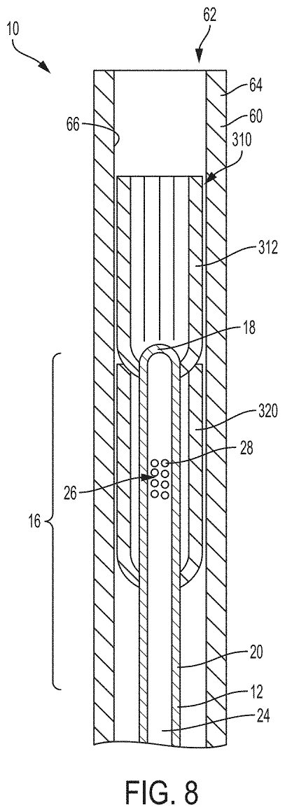

The flanges 312, 320 are capable of transitioning between a retracted or restrained position (shown in FIGS. 6 and 8) and a deployed position (shown in FIG. 7). In the deployed position, the outer edge or rim 318, 326 of each flange 312, 320 extends radially outwardly from the sidewall 20 of the tube 12. In the restrained position, the outer edge or rim 318, 326 of the first flange 312 and/or the second flange 320 are positioned against the sidewall 20 of the tube 12.

In some examples, the flanges 312, 320 comprise a plurality of radially extending cuts or slits 328 extending inwardly from the outer edge or rim 318, 326 of the flanges 312, 320. For example, the cuts or slits 328 may extend through ½, ⅔, ¾, or more of the flange 312, 320. The cuts or slits 328 may be about 0.5 mm to about 2.0 mm (0.020 inch to about 0.080 inch) wide or, preferably, about 1.5 mm (0.060 inch) wide. The cuts or slits 328 separate the flanges into petal portions generally denoted by 330 in FIGS. 3-5. For example, a flange 312, 320 may have from about five to about ten cuts or slits 328 or more. As the flanges 312, 320 are deployed within the bladder, adjacent petal portions 330 separate from one another, such that a distance between adjacent petal portions 330 increases.

In some examples, the flanges 312, 320 may further comprise one or more perforations or holes 332 extending therethrough. The holes 332 can have a variety of shapes including circular, non-circular, elliptical, rectangular, and others. The holes 332 may be identical or different in size and/or shape. In one example, one or more of the holes 332 is circular having a diameter between about 0.25 mm to about 15 mm (0.01 inch and 0.6 inch). As shown in FIGS. 3-5, the holes 332 are arranged in a circle around a central portion of each flange 312, 320. However, other configurations of the holes 332 may also be employed. The holes 332 are positioned to improve distribution of negative and/or positive pressure within the bladder and, in particular, to prevent the flanges 312, 320 from blocking negative and/or positive pressure provided through the drainage lumen 24 from reaching portions of the bladder, ureters, or kidneys.

With continued reference to FIGS. 3-5, the flanges 312, 320 may be identical or may have a different size, shape, and/or material composition. In some examples, the flanges 312, 320 have a convex or dome-shaped distally facing surface configured to be contacted by the bladder wall. In other examples, the flanges 312, 320 may be formed in any shape sufficient for preventing or inhibiting the bladder wall from collapsing into the trigone region and/or occluding the ureteral openings. For example, one or more of the flanges 312, 320 may be a flat disc. In other examples, one or more of the flanges 312, 320 can be a non-circular shape, an elliptical shape, square shaped, polygonal shaped, or any other suitable shape for directing portions of the bladder wall away from the ureter openings. In other examples, one or more of the flanges 312, 320 can have a convex shape, which may, for example, correspond to a curvature of an inferior or lower portion of the bladder wall. Further, it is noted that due to the flexibility of the flanges 312, 320, the flanges 312, 320 may conform to a variety of shapes or configurations when deployed in the bladder. For example, the second flange 320 may initially be convex and, upon contact with the inferior bladder wall, transition to a concave configuration.

The sizes of the flanges 312, 320 can also be identical or different. For example, the outer rim 316, 326 of the flanges 312, 320 can have a maximum outer diameter OD1, OD2 of between about 10 mm and 100 mm (0.3 inch and 4.0 inches) and a thickness T1, T2 ranging from about 0.5 mm to 2.0 mm (0.02 inches and about 0.08 inch). In some examples, the flanges 312, 320 may have a graduated configuration in which the proximal or second flange 320 is larger than the distal or first flange 312. Configurations having a larger or more stable proximal or second flange 320 may be desirable since the proximal or second flange 320 effectively anchors the tissue support 310 of the catheter 10 in the bladder and restricts or inhibits the tissue support 310 from slipping from the bladder when deployed. In such configurations, the maximum outer diameter OD1 of the first flange 312 can be from about 10 mm to 40 mm (0.4 inch to 1.6 inches) and a height H1 of about 7.5 mm to 15 mm (0.3 inch to about 0.6 inch). The outer diameter OD2 of the second flange 320 can be from 25 mm to 50 mm (1.0 inch and about 2.0 inches). A height H2 of the second flange can be about 8 mm to 16 mm.

Delivery Catheter

As shown in FIGS. 6, 7, and 8, in some examples, the catheter device 10 further comprises an outer delivery tube or delivery catheter 60 for assisting in placement of the tissue supporting portion 310 of the device in the patient's bladder. The delivery catheter 60 can be a tubular structure sized to receive the drainage tube 12 and the tissue support 310. The delivery catheter 60 can comprise a proximal end (not shown) configured to be located outside of the body, an open distal end 62 configured to be advanced through the urinary tract to the urethra or bladder, and a sidewall 64 expending therebetween. As was the case with the elongated tube 12, the length of the delivery catheter 60 is variable depending on age and gender of the patient. Generally, a length of a sterile portion of the delivery catheter 60 is about 25 mm to 75 mm (1.0 inch to 3.0 inches) for women, to about 500 mm (20.0 inches) for men. A total length of the delivery catheter 60 including sterile and non-sterile portions can be 50 cm or more (several feet). An inner diameter ID of the delivery catheter 60 is larger than the outer diameter of the drainage tube 12. For example, the delivery catheter 60 may have an inner diameter ID from about 1.0 mm to about 20 mm.

As shown in FIG. 6, prior to deployment, the distal end 18 of the drainage tube 12 and flanges 312, 320 are disposed in a central channel of the delivery catheter 60 in a pre-use restrained configuration. In the pre-use retrained configuration, the flanges 312, 320 are in a dome-shaped or convex orientation, such that a central portion 314, 322 of each flange 312, 320 is distal to the outer edge or rim 318, 326. The flanges 312, 320 contact an inner surface 66 of the sidewall 64 of the delivery catheter 60 to maintain the flanges in the pre-use position.

Once the distal end 62 of the delivery catheter 60 and structures contained therein are positioned in the bladder, a user may deploy the drainage tube 12 and tissue support 310 by advancing the drainage tube 12 and tissue support 310 through the open distal end 62 of the delivery catheter 60. The tissue support 310 is shown deployed from the delivery catheter in FIG. 7. As previously discussed, the flanges 312, 320 can be naturally biased to the deployed position. Accordingly, as each flange 312, 320 advances past the open distal end 62 of the delivery catheter 60, the outer edge(s) or rim(s) 318, 326 of the flange(s) 312, 320 extend radially outwardly from the sidewall 20 of the drainage tube 12. Once both flanges 312, 320 are deployed, the catheter 10 is in position for applying negative pressure to the bladder, ureter(s), and kidney(s) through the drainage lumen of the tube 12. At this point, the flanges 312, 320 and the tube 12 generally float freely within the bladder 110. When the negative pressure is applied to the bladder, the bladder wall is drawn against the flanges 312, 320, and the bladder wall is prevented from occluding the ureters or drainage portion 26 of the drainage tube 12 by the flanges 312, 320. In some instances, the proximally facing surface of the second flange 320 provides a stable base for seating the tissue support 310 and distal portion 16 of the elongated tube 12 within the bladder. A distally facing surface of the first flange 312 contacts and supports portions of the bladder wall to prevent the bladder wall from occluding the ureteral openings. If present, a top or third flange can provide additional support for the superior bladder wall and, in some cases, prevents the bladder from collapsing.

When ready to remove tissue support 310 and distal portion 16 of the tube 12, the user pulls the tube 12 in a proximal direction to draw the distal portion 16 of the tube 12 and tissue support 310 into the delivery catheter 60. As shown in FIG. 8, when retracted into the delivery catheter 60, the flanges 312, 320 are restrained within the deployment catheter 60 in a post-use restrained orientation. In the post-use restrained orientation, the flanges 312, 320 have a concave surface in which the central portions 314, 322 of the flanges 312, 320 are proximal to the outer edges or rims 318, 326 of the flanges 312, 320. Contact between the inner surface 66 of the sidewall 62 of the deployment catheter 60 and the flanges 312, 320 causes the flanges 312, 320 to remain in the retracted position. Once the tissue support 310 and distal portion 16 of the drainage tube 12 are entirely or partially retracted into the delivery catheter 60, the delivery catheter 60 can be removed from the urinary tract by, for example, pulling the delivery catheter 60 and structures contained therein through the urethra and from the body.

Tissue Support with Three Flanges

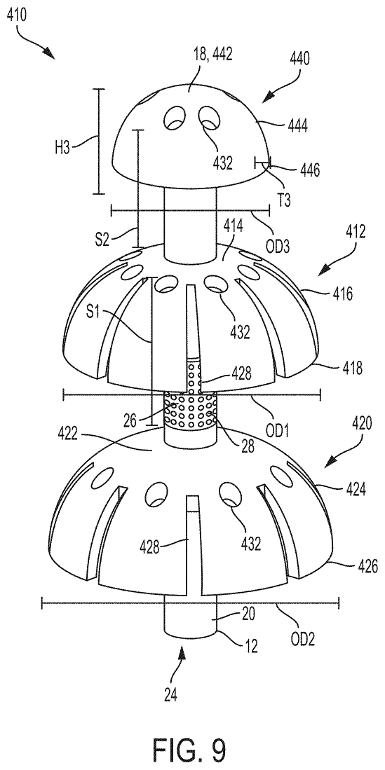

As described herein, a tissue support of a bladder catheter 10 can have varying numbers of flanges in different configurations. In FIG. 9, a tissue support 410 of a bladder catheter 10 is illustrated comprising a middle or first flange 412 positioned between a proximal or second flange 420 and a distal or third flange 440. The flanges 412, 420, 440 can be substantially similar in size and shape to previously described examples and include a central portion 414, 422, 442, an outer rim 418, 426, 446, and an outer portion 416, 424, 444 defining a dome-shaped and/or convex surface extending therebetween.

In some examples, the first and second flanges 412, 420 can be substantially similar in size, shape, and material to the flanges in previously described examples of the bladder catheter 10. For example, the proximal or second flange 420 can be sized to rest against the inferior bladder wall. The middle or first flange 412 can extend above the trigone region and/or ureteral orifices to prevent occlusion of the trigone region and/or ureteral orifices when negative pressure is applied to the bladder.

The distal or third flange 440 can be formed from the same material as the first flange 412 and/or the second flange 420. The third flange 440 may comprise slits 428 and/or holes 432, similar to the first and second flanges 412, 420. The third flange 440 can be configured to support a superior portion of the bladder wall from collapsing against the other flanges and/or against drainage portion(s) 26 of the tube 12. In some examples, since the superior portion of the bladder wall is soft and compliant, the distal or third flange 440 can be formed from a soft material, such as silicone, having similar properties to the middle or first flange 412. When deployed in the bladder, the third flange 440 is positioned farther away from the trigone region and ureter openings than the other flanges 412, 420 and, as such, may contribute less to preventing occlusion of the ureter openings than the other flanges 412, 420. Accordingly, the third flange 440 may be smaller (e.g., may have a smaller maximum outer diameter OD3 or be thinner) than the other flanges 412, 420. For example, the maximum outer diameter OD3 of the outer rim 448 can be about 7.0 mm to about 20 mm (0.3 inch to 0.8 inch) and a thickness T3 of about 0.5 mm to about 2.0 mm (0.02 inch to about 0.08 inch). The third flange 440 can have an outer flange radius or height H3 of from about 3.0 mm to 10 mm (0.12 inch to about 0.4 inch). A distance S2 between the middle or first flange 412 and the third flange 440 can be about 5.0 mm to about 25 mm (0.2 inch to 1.0 inch) or more. In some examples, the third flange 440 is positioned on or covering the distal end 18 of the elongated tube 12, as shown in FIG. 9. In other examples, the distal end 18 of the drainage tube 12 may extend from the third flange, for example, by a distance of about 5.0 mm to about 15 mm (0.2 inch to about 0.6 inch) or more.

Tissue Support with Height Adjustment

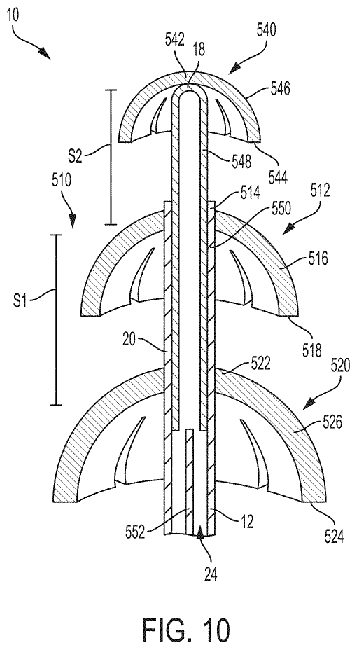

A bladder catheter 10 including a height-adjustable tissue support 510 is shown in FIG. 10. The tissue support 510 includes flanges 512, 520, 540, which are substantially similar in size and configuration to flanges in previously described examples. In particular, the flanges 512, 520, 540 comprise: a central portion 514, 522, 542 connected to the sidewall 20 and/or distal end 18 of the elongated tube 12; an outer rim 518, 526, 546; and an outer portion 516, 524, 544 defining a dome-shaped surface extending between the central portion 514, 522, 542 and the outer rim 518, 526, 546. A distance Si between the first flange 512 and the second flange 520 and/or between the first flange 512 and the third flange 540 can be height adjustable to provide greater flexibility for different sized patients and to provide greater control of positioning of the elongated tube 12 and flanges 512, 520, 540 in the bladder. Various sliding and telescoping mechanisms can be used for adjusting the distance between the flanges 512, 520, 540 within the scope of the present disclosure. For example, one or more of the flanges 512, 520, 540 may be capable of sliding along the sidewall 20 of the elongated tube 12 so that a distance between flanges 512, 520, 540 can be adjusted. In that case, the flanges 512, 520, 540 can include clamping or locking structures for removably engaging the central portion 514, 522, 542 of the flanges 512, 520, 540 to the sidewall 20 of the tube, so that the flanges 512, 520, 540 can be repositioned.

In other examples, as shown in FIG. 10, the distal portion 16 of the elongated tube 12 comprises a telescoping mechanism. For example, the distal portion 16 of the tube 12 can comprise a distal inner tube segment 548 slidably received within a proximal outer tube segment 550. In order to adjust the distance S2 between the first flange 512 and the second flange 540, a user may draw the inner tube segment 548 into the outer or proximal segment 550 using a retraction mechanism, as is known in the art. For example, the inner tube segment 548 may be mounted to a retraction wire 552 extending through a drainage lumen 24 of the tube 12 or through another separate lumen of the elongated tube 12. A user may draw the distal or inner segment 548 into the outer or proximal segment 550 by pulling on the retraction wire 552, thereby decreasing a distance between flanges 512, 540. The user may extend the length of the drainage tube 12 to increase the distance S2 between the flanges 512, 540 by, for example, pushing the wire 552 or a separate push rod through the lumen 24 to extend the distal inner tube segment 548 from the proximal outer tube segment 550. In other examples, the tissue support 510 can comprise a telescoping mechanism between the first flange 512 and the second flange 520 to adjust a distance S1 between the flanges 512, 520. In other examples, the tissue support 510 can include multiple telescoping mechanisms including three or more tube segments to provide greater adjustability for the tissue support 510 and positioning of the flanges 512, 520, 540.

Tissue Support with One Flange

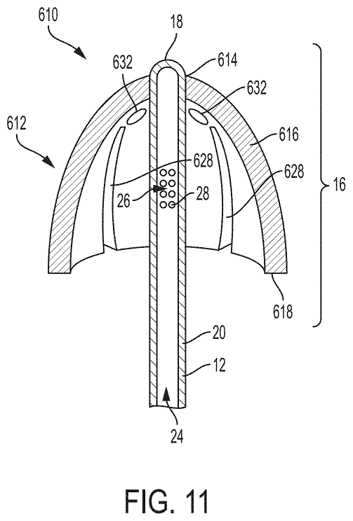

With reference to FIG. 11, a tissue support 610 including a single flange 612 is illustrated. The single flange 612 can be similar in size and shape to the middle flange in previously described examples and can comprise: a central portion 614 covering the distal end 18 of the elongated tube 12; an outer rim 618; and a dome-shaped outer portion 616 extending between the central portion 612 and the outer rim 618. The single flange 612 is positioned on the distal portion 16 of the tube 12 distal to the drainage portion 26. As in previous examples, when deployed, the tissue support 610 defines a three-dimensional shape of sufficient size to permit flow of at least a portion of fluid contained in the bladder from the bladder through the drainage portion 26 of the elongated tube 12 to the at least one drainage lumen 24.

In some examples, the single flange 612 is sized to rest in the bladder at a position distal to the trigone region and ureteral openings. When negative pressure is applied to the bladder, the superior bladder wall is drawn against the distally facing surface of the deployed flange 610. The flange 610 is configured to support the superior bladder wall from occluding the ureteral orifices.

As in previous examples, the single flange 612 comprises slits 628 and/or holes 632 extending through the flange 612 from a proximally facing surface to a distally facing surface thereof. In some instances, fluid, such as urine, may pass through the flange 612 via the slits 628 and/or openings 632 and into the drainage lumen 24 through the drainage portion 26. In other instances, as described above, the ureteral openings can be positioned under or covered by the first flange 612. In that case, fluid entering the bladder through the ureteral openings can be drawn to the drainage portion 26 by the negative pressure without passing through the flange 612.

Ureteral Stents

As discussed above, the urine collection catheters 10 disclosed herein can be used to apply negative pressure therapy to increase renal perfusion. As such, negative pressure delivered through a urine collection catheter 10 deployed in the bladder must transfer through the ureters 116, 118 to the kidneys 112, 114. In some examples, ureteral catheters or stents 30, 32 can be inserted through the ureters 116, 118 to maintain patency of the ureters 116, 118 and to ensure that the ureteral orifices or openings 124, 126 remain open upon application of negative pressure to the bladder.

As used herein, “maintain patency of fluid flow between a kidney and a bladder of the patient” means establishing, increasing or maintaining flow of fluid, such as urine, from the kidneys through the ureter(s), ureteral stent(s) and/or ureteral catheter(s) to the bladder. As used herein, “fluid” means urine and any other fluid from the urinary tract.

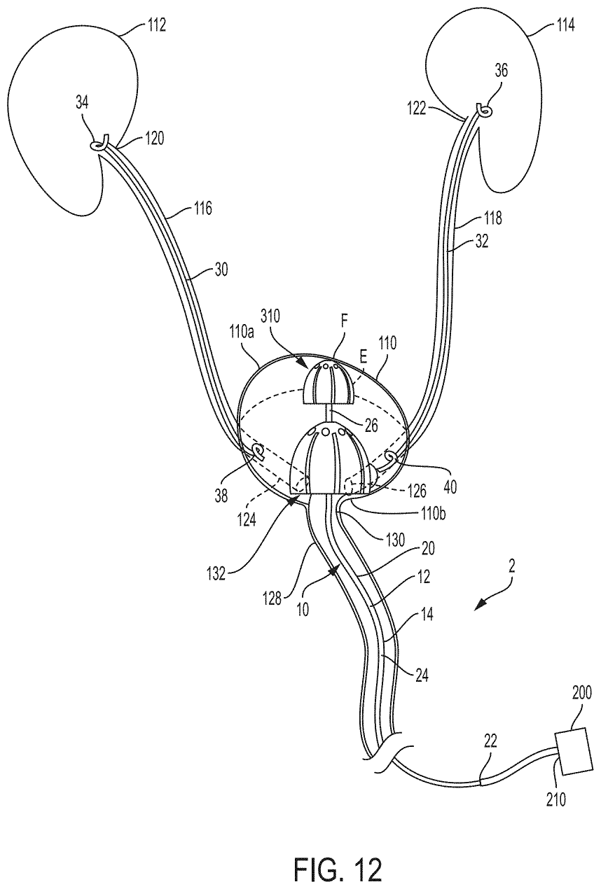

An exemplary urine collection system 2 including the urine collection catheter 10, tissue support 310 deployed in the bladder, and the ureteral stents 30, 32 is shown in FIG. 12. The stent 30 is deployed in the right ureter 116, such that a distal end or retention portion of the stent 30 extends to the right kidney 112, or to the renal pelvis 120 adjacent to the right kidney 112. The ureteral stent 32 is deployed in the left ureter 118, such that a distal end of the stent 32 extends to the left renal pelvis 122 or left kidney 114. Typically, these stents 30, 32 are deployed by inserting a stent having a nitinol wire therethrough through the urethra 128 and bladder 110 up to the kidney(s) 112, 114, then withdrawing the nitinol wire from the stent 30, 32, which permits the stent to assume a deployed configuration. Many of the above stents have a planar loop 34, 36 on the distal end (to be deployed in the kidney), and some also have a planar loop 38, 40 on the proximal end of the stent which is deployed in the bladder 110. When the nitinol wire is removed, the stent assumes the pre-stressed planar loop shape at the distal and/or proximal ends. To remove the stent 30, 32, a nitinol wire is inserted to straighten the stent and the stent is withdrawn from the ureter and urethra.

Some examples of ureteral stents 30, 32 that can be useful in the present systems and methods include CONTOUR™ ureteral stents, CONTOUR VL™ ureteral stents, POLARIS™ Loop ureteral stents, POLARIS™ Ultra ureteral stents, PERCUFLEX™ ureteral stents, PERCUFLEX™ Plus ureteral stents, STRETCH™ VL Flexima ureteral stents, each of which are commercially available from Boston Scientific Corporation of Natick, Mass. See “Ureteral Stent Portfolio”, a publication of Boston Scientific Corp., (July 2010), hereby incorporated by reference herein. The CONTOUR™ and CONTOUR VL™ ureteral stents are constructed with soft Percuflex™ Material that becomes soft at body temperature and is designed for a 365-day indwelling time. Variable length coils on distal and proximal ends allow for one stent to fit various ureteral lengths. The fixed length stent can be 6F-8F with lengths ranging from 20 cm-30 cm, and the variable length stent can be 4.8F-7F with lengths of 22-30 cm. Other examples of suitable ureteral stents include INLAY® ureteral stents, INLAY® OPTIMA® ureteral stents, BARDEX®double pigtail ureteral stents, and FLUORO-4™ silicone ureteral stent, each of which are commercially available from C.R. Bard, Inc. of Murray Hill, N.J. See “Ureteral Stents”, http://www.bardmedical.com/products/kidney-stone-management/ureteral-stents/ (Jan. 21, 2018), hereby incorporated by reference herein.

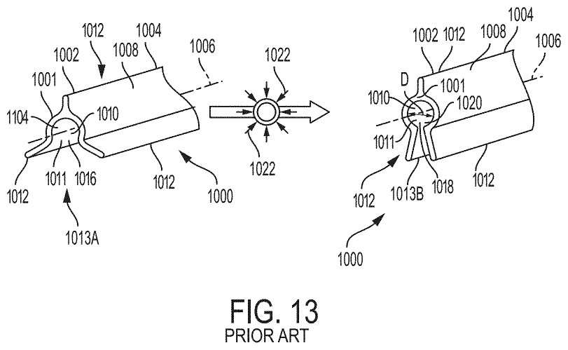

Other examples of suitable ureteral stents 30, 32 are disclosed in PCT Patent Application Publication WO 2017/019974, which is incorporated by reference herein. In some examples, as shown, for example, in FIGS. 1-7 of WO 2017/019974 and in FIG. 13 herein (same as FIG. 1 of WO 2017/019974), a ureteral stent 1000 can comprise: an elongated body 1001 comprising a proximal end 1002, a distal end 1004, a longitudinal axis 1006, an outer surface 1008, and an inner surface 1010, wherein the inner surface 1010 defines a transformable bore 1011 that extends along the longitudinal axis 1006 from the proximal end 1002 to the distal end 1004; and at least two fins 1012 projecting radially away from the outer surface 1008 of the body 1001; wherein the transformable bore 1011 comprises: (a) a default orientation 1013A (shown on the left in FIG. 13) comprising an open bore 1014 defining a longitudinally open channel 1016; and (b) a second orientation 1013B (shown on the right in FIG. 13) comprising an at least essentially closed bore 1018 or closed bore defining a longitudinally essentially closed drainage channel 1020 along the longitudinal axis 1006 of the elongated body 1001, wherein the transformable bore 1011 is moveable from the default orientation 1013A to the second orientation 1013B upon radial compression forces 1022 being applied to at least a portion of the outer surface 1008 of the body 1001.

In some examples, as shown in FIG. 13, the drainage channel 1020 of the ureteral stent 1000 has a diameter D which is reduced upon the transformable bore 1011 moving from the default orientation 1013A to the second orientation 1013B, wherein the diameter is reducible up to the point above where urine flow through the transformable bore 1011 would be reduced. In some examples, the diameter D is reduced by up to about 40% upon the transformable bore 1011 moving from the default orientation 1013A to the second orientation 1013B. In some examples, the diameter D in the default orientation 1013A can range from about 0.75 to about 5.5 mm, or about 1.3 mm or about 1.4 mm. In some examples, the diameter D in the second orientation 1013B can range from about 0.4 to about 4 mm, or about 0.9 mm.

In some examples, one or more fins 1012 comprise a flexible material that is soft to medium soft based on the Shore hardness scale. In some examples, the body 1001 comprises a flexible material that is medium hard to hard based on the Shore hardness scale. In some examples, one or more fins have a durometer between about 15 A to about 40 A. In some examples, the body 1001 has a durometer between about 80 A to about 90 A. In some examples, one or more fins 1012 and the body 1001 comprise a flexible material that is medium soft to medium hard based on the Shore hardness scale, for example having a durometer between about 40 A to about 70 A.

In some examples, one or more fins 1012 and the body 1001 comprise a flexible material that is medium hard to hard based on the Shore hardness scale, for example having a durometer between about 85 A to about 90 A.

In some examples, the default orientation 1013A and the second orientation 1013B support fluid or urine flow around the outer surface 1008 of the stent 1000 in addition to through the transformable bore 1011.

In some examples, one or more fins 1012 extend longitudinally from the proximal end 1002 to the distal end 1004. In some examples, the stent has two, three or four fins.

In some examples, the outer surface 1008 of the body has an outer diameter in the default orientation 1013A ranging from about 0.8 mm to about 6 mm, or about 3 mm. In some examples, the outer surface 1008 of the body has an outer diameter in the second orientation 1013B ranging from about 0.5 mm to about 4.5 mm, or about 1 mm. In some examples, one or more fins have a width or tip ranging from about 0.25 mm to about 1.5 mm, or about 1 mm, projecting from the outer surface 1008 of the body in a direction generally perpendicular to the longitudinal axis.

In some examples, the radial compression forces are provided by at least one of normal ureter physiology, abnormal ureter physiology, or application of any external force. In some examples, the ureteral stent 1000 purposefully adapts to a dynamic ureteral environment, the ureteral stent 1000 comprising: an elongated body 1001 comprising a proximal end 1002, a distal end 1004, a longitudinal axis 1006, an outer surface 1008, and an inner surface 1010, wherein the inner surface 1010 defines a transformable bore 1011 that extends along the longitudinal axis 1006 from the proximal end 1002 to the distal end 1004; wherein the transformable bore 1011 comprises: (a) a default orientation 113A comprising an open bore 114 defining a longitudinally open channel 116; and (b) a second orientation 1013B comprising an at least essentially closed bore 1018 defining a longitudinally essentially closed channel 1020, wherein the transformable bore is moveable from the default orientation 1013A to the second orientation 1013B upon radial compression forces 1022 being applied to at least a portion of the outer surface 1008 of the body 1001, wherein the inner surface 1010 of the body 1001 has a diameter D which is reduced upon the transformable bore 1011 moving from the default orientation 1013A to the second orientation 1013B, wherein the diameter is reducible up to the point above where fluid flow through the transformable bore 1011 would be reduced. In some examples, the diameter D is reduced by up to about 40% upon the transformable bore 1011 moving from the default orientation 1013A to the second orientation 1013B.

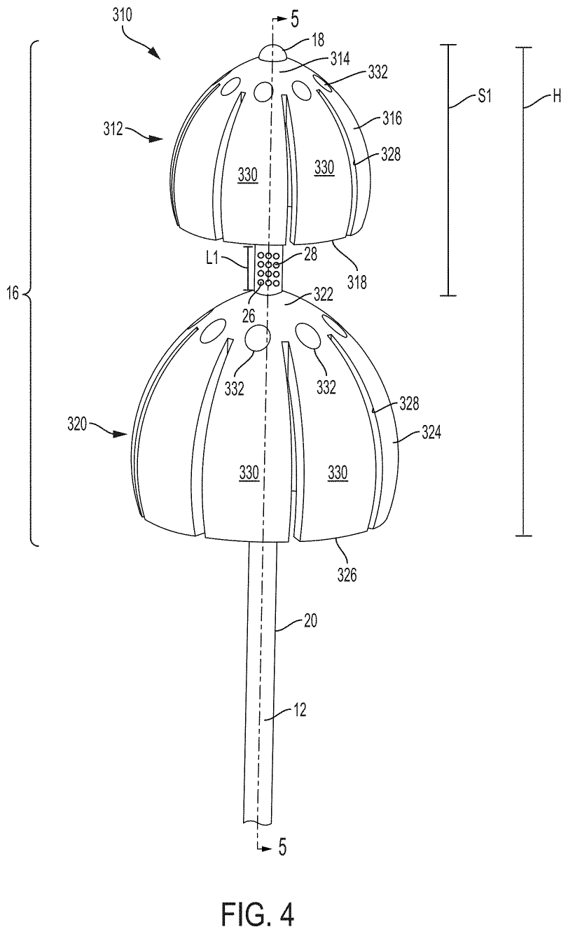

Other examples of suitable ureteral stents are disclosed in United States Patent Application Publication No. 2002/0183853 A1, which is incorporated by reference herein. In some examples, as shown, for example, in FIGS. 4, 5 and 7 of US 2002/0183853 A1 and in FIGS. 3-5 herein (same as FIGS. 1 of 4, 5 and 7 of US 2002/0183853 A1), the ureteral stent comprises an elongated, body 10 comprising a proximal end 12, a distal end 14 (not shown) , a longitudinal axis 15, and at least one drainage channel (for example, 26, 28, 30 in FIGS. 4; 32, 34, 36, and 38 in FIGS. 5; and 48 in FIG. 6) that extends along the longitudinal axis 15 from the proximal end 12 to the distal end 14 to maintain patency of fluid flow between a kidney and a bladder of the patient. In some examples, the at least one drainage channel is partially open along at least a longitudinal portion thereof. In some examples, the at least one drainage channel is closed along at least a longitudinal portion thereof. In some examples, the at least one drainage channel is closed along the longitudinal length thereof. In some examples, the ureteral stent is radially compressible. In some examples, the ureteral stent is radially compressible to narrow the at least one drainage channel. In some examples, the elongated body 10 comprises at least one external fin 40 along the longitudinal axis 15 of the elongated body 10. In some examples, the elongated body comprises one to four drainage channels. The diameter of the drainage channel can be the same as described above.

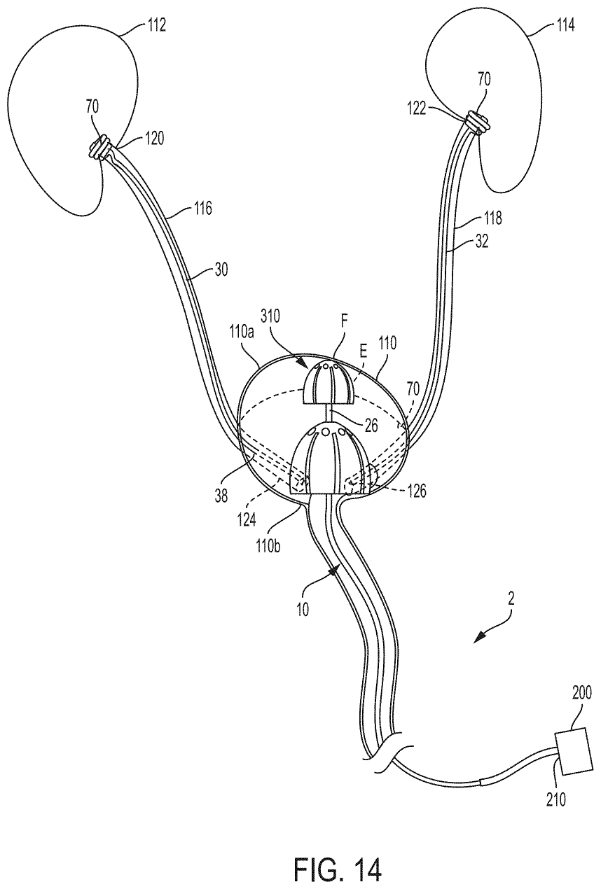

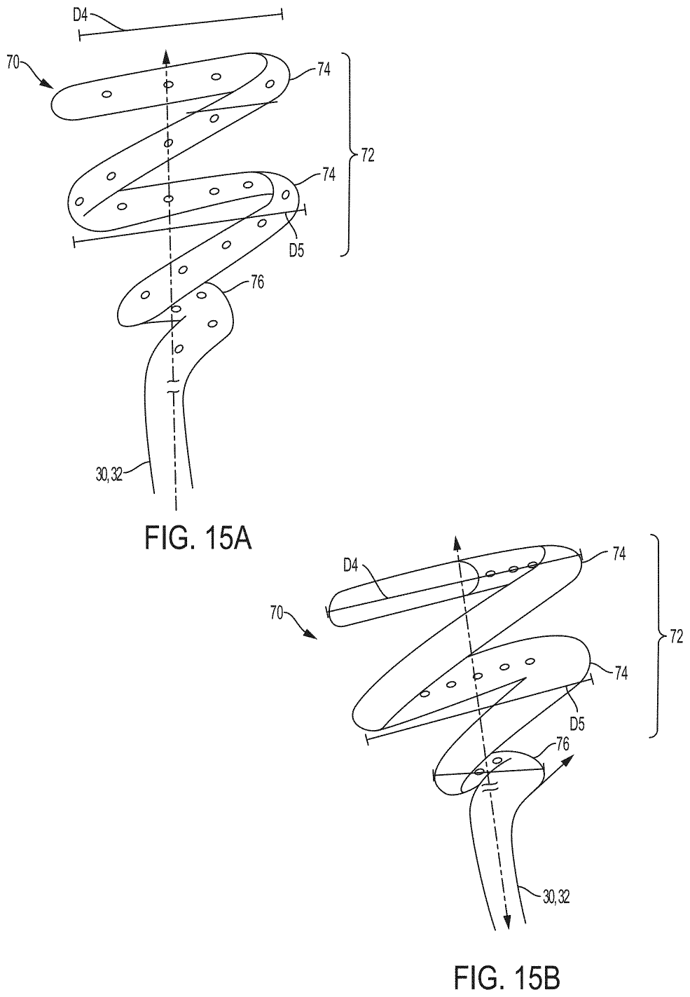

With reference to FIG. 14, other embodiments of exemplary ureteral stents 30, 32 which can be used within the scope of the present disclosure to maintain patency of fluid from the kidneys 112, 114 and the ureters 116, 118 to the bladder 110 comprises an elongated tube, which extends from a retention portion located in the bladder. The stents 30, 32 include a helical retention portion 70 comprising a plurality of coils 72 (shown in FIGS. 15A and 15B) which maintains a distal end the tube in a desired position in a patient's renal pelvis 120, 122 or kidney 112, 114. For example, as described in further detail in connection with FIGS. 15A and 15B, the helical retention portion 70 can comprise at least a first coil having a first diameter; at least a second coil having a second diameter, the first diameter being less than the second diameter, the second coil being closer to an end of the distal portion of the drainage lumen than the first coil; and one or more perforations on a sidewall of the coiled retention portion of the distal portion of the drainage lumen for permitting fluid flow into the drainage lumen. In some examples, the helical stents 30, 32 could be configured such that, prior to insertion into a patient's urinary tract, a portion of the drainage lumen that is proximal to the retention portion defines a straight or curvilinear central axis, and wherein, when deployed, the first coil and the second coil of the retention portion extend about an axis of the retention portion that is at least partially coextensive with the straight or curvilinear central axis of the portion of the drainage lumen.

As shown in FIGS. 15A and 15B, exemplary helical retention portions 70 comprising a plurality of helical coils, which can be used to anchor the ureteral stents disclosed herein are illustrated. The retention portions 70 generally comprise one or more full coils 74 and one or more half or partial coils 76. The retention portion 70 is capable of moving between a contracted position and the deployed position with the plurality of helical coils. For example, a substantially straight guidewire can be inserted through the retention portion 70 to maintain the retention portion 70 in a substantially straight contracted position. When the guidewire is removed, the retention portion 70 can transition to its coiled configuration. In some examples, the coils 74, 76 extend radially and longitudinally from the distal portion 16 of the tube 12. In a preferred exemplary embodiment, the retention portion 70 comprises two full coils 74 and one half coil 76. The outer diameter of the full coils 74, shown by line D4, can be about 18±2 mm. The half coil 76 diameter D5 can be about 14 mm±2 mm. The retention portion 70 can further comprise the one or more drainage holes, such as perforations 28, configured to draw fluid into an interior of the elongated tube 412 of the stent.

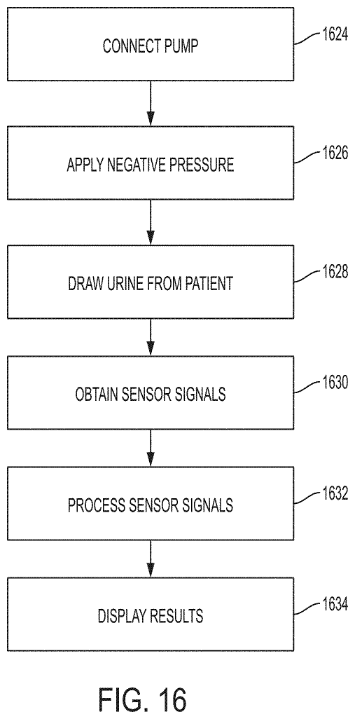

With reference to FIG. 16, steps for using the urine collection assembly for inducement of negative pressure in the ureter(s) and/or kidney(s) are illustrated. As shown at box 1624, after the indwelling portions of the bladder and/or ureteral catheters are correctly positioned and anchoring/retention structures are deployed, the external proximal ends of the catheter(s) are connected to fluid collection or pump assemblies. For example, the ureteral catheter(s) can be connected to a pump for inducing negative pressure at the patient's renal pelvis and/or kidney. In a similar manner, the bladder catheter can be connected directly to a urine collection container for gravity drainage of urine from the bladder or connected to a pump for inducing negative pressure at the bladder.

Once the catheter(s) and pump assembly are connected, negative pressure can be applied to the catheter as shown at box 1626. The negative pressure collapses the bladder, thereby drawing portions of the bladder wall against the outer portion of the flanges, but not within the deployed dome. It is critical to maintain a continuous fluid column between the pump and the kidney(s) through which the pressure differential is established. Therefore, in one embodiment, the flanges are configured to open and orient over the entire trigone region, such that both ureteral orifices and the urethra are contained within the deployed dome and the fluid pathway is maintained between the urethra and ureteral orifices. In this scenario, a negative pressure applied to the bladder catheter results in an equivalent negative pressure delivery to both ureters for transmission to both kidneys. In other embodiments, the negative pressure may be regulated so that one kidney receives a different level of negative pressure than a contralateral kidney. If there is a fluid source at one end of a fluid column and a negative pressure is applied to another end of this fluid column, fluid flow through that column can be increased in a direction away from the relatively higher pressure fluid source and toward the negative pressure source. Delivery of a negative pressure to the renal calyces takes advantage of the higher fluid pressure that is upstream of the filtrate produced in the kidneys, pushing the filtrate towards the negative pressure source and increasing the glomerular filtration rate. This increased flow of filtrate through the tubules can also decrease the reabsorption potential for sodium and water further contributing to greater urine production.

In some examples, mechanical stimulation can be provided to portions of the ureters and/or renal pelvis to supplement or modify therapeutic affects obtained by application of negative pressure. For example, mechanical stimulation devices, such as linear actuators and other known devices for providing, for example, vibration waves, disposed in distal portions of the ureteral catheter(s) can be actuated. While not intending to be bound by theory, it is believed that such stimulation effects adjacent tissues by, for example, stimulating nerves and/or actuating peristaltic muscles associated with the ureter(s) and/or renal pelvis. Stimulation of nerves and activation of muscles may produce changes in pressure gradients or pressure levels in surrounding tissues and organs which may contribute to or, in some cases, enhance therapeutic benefits of negative pressure therapy. In some examples, the mechanical stimulation can comprise pulsating stimulation. In other examples, low levels of mechanical stimulation can be provided continuously as negative pressure is being provided through the ureteral catheter(s). In other examples, inflatable portions of the ureteral catheter could be inflated and deflated in a pulsating manner to stimulate adjacent nerve and muscle tissue, in a similar manner to actuation of the mechanical stimulation devices described herein.

As a result of the applied negative pressure, as shown at box 1628, urine is drawn into the catheter at the plurality of drainage ports or openings at the distal end thereof, through the drainage lumen of the catheter, and to a fluid collection container for disposal. As the urine is being drawn to the collection container, at box 1630, sensors disposed in the fluid collection system provide a number of measurements about the urine that can be used to assess the volume of urine collected, as well as information about the physical condition of the patient and composition of the urine produced. In some examples, the information obtained by the sensors is processed, as shown at box 1632, by a processor associated with the pump and/or with another patient monitoring device and, at box 1634, is displayed to the user via a visual display of an associated feedback device.

### Exemplary Fluid Collection System

Having described an exemplary urine collection devices, systems, and method of positioning such an assembly in the patient's body, with reference to FIG. 17, a system 700 for inducing negative pressure to a patient's ureter(s) and/or kidney(s) will now be described. The system 700 can comprise the ureteral catheter(s), bladder catheter or the urine collection assembly 100 described hereinabove. As shown in FIG. 17, urine collection catheter 10 comprising the tissue support 310 is connected to one or more fluid collection containers 712 for collecting urine drawn from the renal pelvis and/or bladder. The fluid collection container 712 connected to the urine collection catheter 10 can be in fluid communication with an external fluid pump 710 for generating negative pressure in the ureter(s) and kidney(s) through the catheter 10. The pump 710 can be the pump 200 shown in FIG. 2. In other examples, the pump 710 can be a vacuum pump, rotary pump, or other negative pressure source, as is known in the art. As discussed herein, such negative pressure can be provided for overcoming interstitial pressure and forming urine in the kidney or nephron. In some examples, a connection between the fluid collection container 712 and pump 710 can comprise a fluid lock or fluid barrier to prevent air from entering the renal pelvis or kidney in case of incidental therapeutic or non-therapeutic pressure changes. For example, inflow and outflow ports of the fluid container can be positioned below a fluid level in the container. Accordingly, air is prevented from entering medical tubing or the catheter through either the inflow or outflow ports of the fluid container 712. As discussed previously, external portions of the tubing extending between the fluid collection container 712 and the pump 710 can include one or more filters to prevent urine and/or particulates from entering the pump 710.

As shown in FIG. 17, the system 700 further comprises a controller 714, such as a microprocessor, electronically coupled to the pump 710 and having or associated with computer readable memory 716. In some examples, the memory 716 comprises instructions that, when executed, cause the controller 714 to receive information from sensors 774 located on or associated with portions of the catheter 10. Information about a condition of the patient can be determined based on information from the sensors 774. Information from the sensors 774 can also be used to determine and implement operating parameters for the pump 710.

In some examples, the controller 714 is incorporated in a separate and remote electronic device in communication with the pump 710, such as a dedicated electronic device, computer, tablet PC, or smart phone. Alternatively, the controller 714 can be included in the pump 710 and, for example, can control both a user interface for manually operating the pump 710, as well as system functions such as receiving and processing information from the sensors 774.

The controller 714 is configured to receive information from the one or more sensors 774 and to store the information in the associated computer-readable memory 716. For example, the controller 714 can be configured to receive information from the sensor 774 at a predetermined rate, such as once every second, and to determine a conductance based on the received information. In some examples, the algorithm for calculating conductance can also include other sensor measurements, such as urine temperature, to obtain a more robust determination of conductance.

The controller 714 can also be configured to calculate patient physical statistics or diagnostic indicators that illustrate changes in the patient's condition over time. For example, the system 700 can be configured to identify an amount of total sodium excreted. The total sodium excreted may be based, for example, on a combination of flow rate and conductance over a period of time.

With continued reference to FIG. 17, the system 700 can further comprise a feedback device 720, such as a visual display or audio system, for providing information to the user. In some examples, the feedback device 720 can be integrally formed with the pump 710. Alternatively, the feedback device 720 can be a separate dedicated or a multipurpose electronic device, such as a computer, laptop computer, tablet PC, smart phone, or other handheld electronic devices. The feedback device 720 is configured to receive the calculated or determined measurements from the controller 714 and to present the received information to a user via the feedback device 720. For example, the feedback device 720 may be configured to display current negative pressure (in mmHg) being applied to the urinary tract. In other examples, the feedback device 720 is configured to display current flow rate of urine, temperature, current conductance in mS/m of urine, total urine produced during the session, total sodium excreted during the session, other physical parameters, or any combination thereof.

In some examples, the feedback device 720 further comprises a user interface module or component that allows the user to control operation of the pump 710. For example, the user can engage or turn off the pump 710 via the user interface. The user can also adjust pressure applied by the pump 710 to achieve a greater magnitude or rate of sodium excretion and fluid removal.

Optionally, the feedback device 720 and/or pump 710 further comprise a data transmitter 722 for sending information from the device 720 and/or pump 710 to other electronic devices or computer networks. The data transmitter 722 can utilize a short-range or long-range data communications protocol. An example of a short-range data transmission protocol is Bluetooth®. Long-range data transmission networks include, for example, Wi-Fi or cellular networks. The data transmitter 722 can send information to a patient's physician or caregiver to inform the physician or caregiver about the patient's current condition. Alternatively, or in addition, information can be sent from the data transmitter 722 to existing databases or information storage locations, such as, for example, to include the recorded information in a patient's electronic health record (EHR).

With continued reference to FIG. 17, in addition to the urine sensors 774, in some examples, the system 700 further comprises one or more patient monitoring sensors 724. Patient monitoring sensors 724 can include invasive and non-invasive sensors for measuring information about the patient's urine composition, as discussed in detail above, blood composition (e.g., hematocrit ratio, analyte concentration, protein concentration, creatinine concentration) and/or blood flow (e.g., blood pressure, blood flow velocity). Hematocrit is a ratio of the volume of red blood cells to the total volume of blood. Normal hematocrit is about 25% to 40%, and preferably about 35% and 40% (e.g., 35% to 40% red blood cells by volume and 60% to 65% plasma).

Non-invasive patient monitoring sensors 724 can include pulse oximetry sensors, blood pressure sensors, heart rate sensors, and respiration sensors (e.g., a capnography sensor). Invasive patient monitoring sensors 724 can include invasive blood pressure sensors, glucose sensors, blood velocity sensors, hemoglobin sensors, hematocrit sensors, protein sensors, creatinine sensors, and others. In still other examples, sensors may be associated with an extracorporeal blood system or circuit and configured to measure parameters of blood passing through tubing of the extracorporeal system. For example, analyte sensors, such as capacitance sensors or optical spectroscopy sensors, may be associated with tubing of the extracorporeal blood system to measure parameter values of the patient's blood as it passes through the tubing. The patient monitoring sensors 724 can be in wired or wireless communication with the pump 710 and/or controller 714.

In some examples, the controller 714 is configured to cause the pump 710 to provide treatment for a patient based information obtained from the urine analyte sensor 774 and/or patient monitoring sensors 724, such as blood monitoring sensors. For example, pump 710 operating parameters can be adjusted based on changes in the patient's blood hematocrit ratio, blood protein concertation, creatinine concentration, urine output volume, urine protein concentration (e.g., albumin), and other parameters. For example, the controller 714 can be configured to receive information about a blood hematocrit ratio or creatinine concentration of the patient from the patient monitoring sensors 724 and/or analyte sensors 774. The controller 714 can be configured to adjust operating parameters of the pump 710 based on the blood and/or urine measurements. In other examples, hematocrit ratio may be measured from blood samples periodically obtained from the patient. Results of the tests can be manually or automatically provided to the controller 714 for processing and analysis.

As discussed herein, measured hematocrit values for the patient can be compared to predetermined threshold or clinically acceptable values for the general population. Generally, hematocrit levels for females are lower than for males. In other examples, measured hematocrit values can be compared to patient baseline values obtained prior to a surgical procedure. When the measured hematocrit value is increased to within the acceptable range, the pump 710 may be turned off ceasing application of negative pressure to the ureter or kidneys. In a similar manner, the intensity of negative pressure can be adjusted based on measured parameter values. For example, as the patient's measured parameters begin to approach the acceptable range, intensity of negative pressure being applied to the ureter and kidneys can be reduced. In contrast, if an undesirable trend (e.g., a decrease in hematocrit value, urine output rate, and/or creatinine clearance) is identified, the intensity of negative pressure can be increased in order to produce a positive physiological result. For example, the pump 710 may be configured to begin by providing a low level of negative pressure (e.g., between about 0.10 mmHg and 10 mmHg). The negative pressure may be incrementally increased until a positive trend in patient creatinine level is observed. However, generally, negative pressure provided by the pump 710 will not exceed about 150 mmHg.

With reference to FIGS. 18A and 18B, an exemplary pump 710 for use with the system is illustrated. In some examples, the pump 710 is a micro-pump configured to draw fluid from the catheter(s) 10 and having a sensitivity or accuracy of about 10 mmHg or less. Desirably, the pump 710 is capable of providing a range of flow of urine between 0.05 ml/min and 3 ml/min for extended periods of time, for example, for about 8 hours to about 24 hours per day, for one (1) to about 30 days or longer. At 0.2 ml/min, it is anticipated that about 300 mL of urine per day is collected by the system 700. The pump 710 can be configured to provide a negative pressure to the bladder of the patient, the negative pressure ranging between about 0.1 mmHg and 150 mmHg or about 5 mmHg to about 20 mmHg (gauge pressure at the pump 710). For example, a micro-pump manufactured by Langer Inc. (Model BT100-2J) can be used with the presently disclosed system 700. Diaphragm aspirator pumps, as well as other types of commercially available pumps, can also be used for this purpose. Peristaltic pumps can also be used with the system 700. In other examples, a piston pump, vacuum bottle, or manual vacuum source can be used for providing negative pressure. In other examples, the system can be connected to a wall suction source, as is available in a hospital, through a vacuum regulator for reducing negative pressure to therapeutically appropriate levels.

In some examples, at least a portion of the pump assembly can be positioned within the patient's urinary tract, for example within the bladder. For example, the pump assembly can comprise a pump module and a control module coupled to the pump module, the control module being configured to direct motion of the pump module. At least one (one or more) of the pump module, the control module, or the power supply may be positioned within the patient's urinary tract. The pump module can comprise at least one pump element positioned within the fluid flow channel to draw fluid through the channel. Some examples of suitable pump assemblies, systems and methods of use are disclosed in U.S. Patent Application No. 62/550,259, entitled “Indwelling Pump for Facilitating Removal of Urine from the Urinary Tract”, filed concurrently herewith, which is incorporated by reference herein in its entirety.

In some examples, the pump 710 is configured for extended use and, thus, is capable of maintaining precise suction for extended periods of time, for example, for about 8 hours to about 24 hours per day, for 1 to about 30 days or longer. Further, in some examples, the pump 710 is configured to be manually operated and, in that case, includes a control panel 718 that allows a user to set a desired suction value. The pump 710 can also include a controller or processor, which can be the same controller that operates the system 700 or can be a separate processor dedicated for operation of the pump 710. In either case, the processor is configured for both receiving instructions for manual operation of the pump and for automatically operating the pump 710 according to predetermined operating parameters. Alternatively, or in addition, operation of the pump 710 can be controlled by the processor based on feedback received from the plurality of sensors associated with the catheter.

In some examples, the processor is configured to cause the pump 710 to operate intermittently. For example, the pump 710 may be configured to emit pulses of negative pressure followed by periods in which no negative pressure is provided. In other examples, the pump 710 can be configured to alternate between providing negative pressure and positive pressure to produce an alternating flush and pump effect. For example, a positive pressure of about 0.1 mmHg to 20 mmHg, and preferably about 5 mmHg to 20 mmHg can be provided followed by a negative pressure ranging from about 0.1 mmHg to 50 mmHg.

Treatment for Removing Excess Fluid from a Patient with Hemodilution

According to another aspect of the disclosure, a method for removing excess fluid from a patient with hemodilution is provided. In some examples, hemodilution can refer to an increase in a volume of plasma in relation to red blood cells and/or a reduced concentration of red blood cells in circulation, as may occur when a patient is provided with an excessive amount of fluid. The method can involve measuring and/or monitoring patient hematocrit levels to determine when hemodilution has been adequately addressed. Low hematocrit levels are a common post-surgical or post-trauma condition that can lead to undesirable therapeutic outcomes. As such, management of hemodilution and confirming that hematocrit levels return to normal ranges is a desirable therapeutic result for surgical and post-surgical patient care.

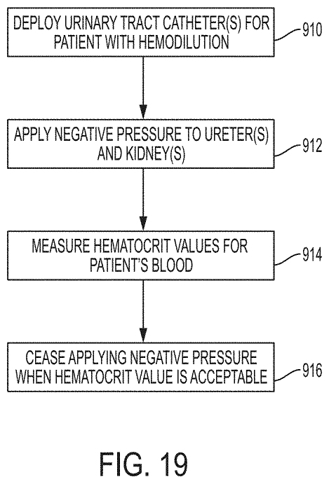

Steps for removing excess fluid from a patient using the devices and systems described herein are illustrated in FIG. 19. As shown in FIG. 19, the treatment method comprises deploying a urinary tract catheter, such as the urine collection catheter configured to be deployed in the bladder or renal pelvis of the present invention, in the ureter and/or kidney of a patient such that flow of urine from the ureter and/or kidney, as shown at box 910. The catheter may be placed to avoid occluding the ureter and/or kidney. In some examples, a fluid collecting portion of the catheter may be positioned in the renal pelvis of the patient's kidney. In some examples, a ureter catheter may be positioned in each of the patient's kidneys. In other examples, a urine collection catheter may be deployed in the bladder or ureter. In some examples, the ureteral catheter comprises one or more of any of the retention portions described herein. For example, the ureteral catheter can comprise a tube defining a drainage lumen comprising a helical retention portion and a plurality of drainage ports. In other examples, the catheter can include an inflatable retention portion (e.g., a balloon catheter), funnel-shaped fluid collection and retention portion, or a pigtail coil.

As shown at box 912, the method further comprises applying negative pressure to the ureter and/or kidney through the catheter to induce production of urine in the kidney(s) and to extract urine from the patient. Desirably, negative pressure is applied for a period of time sufficient to reduce the patient's blood creatinine levels by a clinically significant amount.

Negative pressure may continue to be applied for a predetermined period of time. For example, a user may be instructed to operate the pump for the duration of a surgical procedure or for a time period selected based on physiological characteristics of the patient. In other examples, patient condition may be monitored to determine when sufficient treatment has been provided. For example, as shown at box 914, the method may further comprise monitoring the patient to determine when to cease applying negative pressure to the patient's ureter and/or kidneys. In a preferred and non-limiting example, a patient's hematocrit level is measured. For example, patient monitoring devices may be used to periodically obtain hematocrit values. In other examples, blood samples may be drawn periodically to directly measure hematocrit. In some examples, concentration and/or volume of urine expelled from the body through the catheter may also be monitored to determine a rate at which urine is being produced by the kidneys. In a similar manner, expelled urine output may be monitored to determine protein concentration and/or creatinine clearance rate for the patient. Reduced creatinine and protein concentration in urine may be indicative of over-dilution and/or depressed renal function. Measured values can be compared to the predetermined threshold values to assess whether negative pressure therapy is improving patient condition, and should be modified or discontinued. For example, as discussed herein, a desirable range for patient hematocrit may be between 25% and 40%. In other preferred and non-limiting examples, as described herein, patient body weight may be measured and compared to a dry body weight. Changes in measured patient body weight demonstrate that fluid is being removed from the body. As such, a return to dry body weight represents that hemodilution has been appropriately managed and the patient is not over-diluted.

As shown at box 916, a user may cause the pump to cease providing negative pressure therapy when a positive result is identified. In a similar manner, patient blood parameters may be monitored to assess effectiveness of the negative pressure being applied to the patient's kidneys. For example, a capacitance or analyte sensor may be placed in fluid communication with tubing of an extracorporeal blood management system. The sensor may be used to measure information representative of blood protein, oxygen, creatinine, and/or hematocrit levels. Measured blood parameter values may be measured continuously or periodically and compared to various threshold or clinically acceptable values. Negative pressure may continue to be applied to the patient's kidney or ureter until a measured parameter value falls within a clinically acceptable range. Once a measured values fails within the threshold or clinically acceptable range, as shown at box 916, application of negative pressure may cease.

### Treatment of Patients Undergoing a Fluid Resuscitation Procedure

According to another aspect of the disclosure, a method for removing excess fluid for a patient undergoing a fluid resuscitation procedure, such as coronary graft bypass surgery, by removing excess fluid from the patient is provided. During fluid resuscitation, solutions such as saline solutions and/or starch solutions, are introduced to the patient's bloodstream by a suitable fluid delivery process, such as an intravenous drip. For example, in some surgical procedures, a patient may be supplied with between 5 and 10 times a normal daily intake of fluid. Fluid replacement or fluid resuscitation can be provided to replace bodily fluids lost through sweating, bleeding, dehydration, and similar processes. In the case of a surgical procedure such as coronary graft bypass, fluid resuscitation is provided to help maintain a patient's fluid balance and blood pressure within an appropriate rate. Acute kidney injury (AKI) is a known complication of coronary artery graft bypass surgery. AKI is associated with a prolonged hospital stay and increased morbidity and mortality, even for patients who do not progress to renal failure. See Kim, et al., Relationship between a perioperative intravenous fluid administration strategy and acute kidney injury following off-pump coronary artery bypass surgery: an observational study, Critical Care 19:350 (1995). Introducing fluid to blood also reduces hematocrit levels which has been shown to further increase mortality and morbidity. Research has also demonstrated that introducing saline solution to a patient may depress renal functional and/or inhibit natural fluid management processes. As such, appropriate monitoring and control of renal function may produce improved outcomes and, in particular, may reduce post-operative instances of AKI.

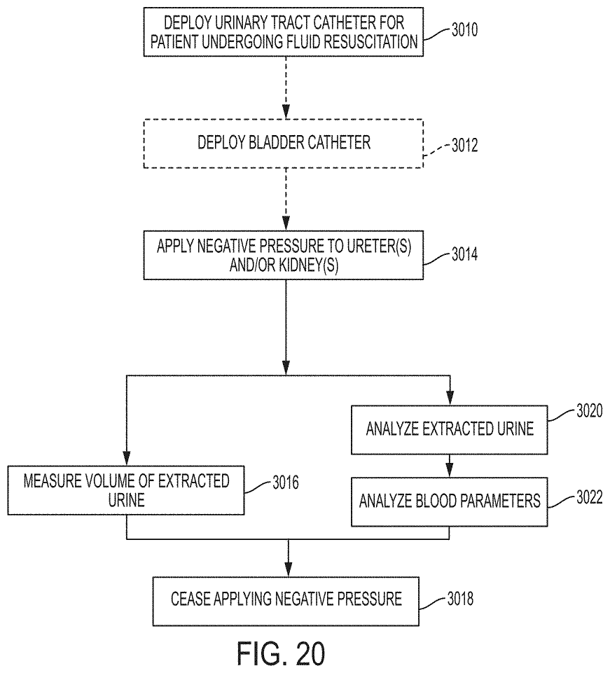

A method of treating a patient undergoing fluid resuscitation is illustrated in FIG. 20. As shown at box 3010, the method comprises deploying a ureteral catheter in the ureter and/or kidney of a patient such that flow of urine from the ureter and/or kidney is not prevented by occlusion of the ureter and/or kidney. For example, a fluid collecting portion of the catheter may be positioned in the renal pelvis. In other examples, the catheter is deployed in the bladder. The catheter can comprise one or more of the urine collection catheters configured to be deployed in the bladder or renal pelvis as described herein. For example, the catheter can comprise a tube defining a drainage lumen and comprising a helical retention portion and a plurality of drainage ports. In other examples, the catheter can include an inflatable retention portion (e.g., a balloon catheter) or a pigtail coil.

As shown at box 3012, optionally, a bladder catheter may also be deployed in the patient's bladder. For example, the bladder catheter may be positioned to seal the urethra opening to prevent passage of urine from the body through the urethra. The bladder catheter can include an inflatable anchor (e.g., a Foley catheter) for maintaining the distal end of the catheter in the bladder. As described herein, other arrangements of coils, helices, and flanges may also be used to obtain proper positioning of the bladder catheter. The bladder catheter can be configured to collect urine which entered the patient's bladder prior to placement of ureteral catheter(s). The bladder catheter may also collect urine which flows past the fluid collection portion(s) of the ureteral catheter and enters the bladder. In some examples, a proximal portion of the ureteral catheter may be positioned in a drainage lumen of the bladder catheter. In a similar manner, the bladder catheter may be advanced into the bladder using the same guidewire used for positioning of the ureteral catheter(s). In some examples, negative pressure may be provided to the bladder through the drainage lumen of the bladder catheter. In other examples, negative pressure may only be applied to the ureteral catheter(s). In that case, the bladder catheter drains by gravity.

As shown at box 3014, following deployment of the ureteral catheter(s) and/or bladder catheter, negative pressure is applied to the ureter and/or kidney through the ureteral catheter(s) and/or bladder catheter. For example, negative pressure can be applied for a period of time sufficient to extract urine comprising a portion of the fluid provided to the patient during the fluid resuscitation procedure. As described herein, negative pressure can be provided by an external pump connected to a proximal end or port of the catheter. The pump can be operated continually or periodically dependent on therapeutic requirements of the patient. In some cases, the pump may alternate between applying negative pressure and positive pressure.

Negative pressure may continue to be applied for a predetermined period of time. For example, a user may be instructed to operate the pump for the duration of a surgical procedure or for a time period selected based on physiological characteristics of the patient. In other examples, patient condition may be monitored to determine when a sufficient amount of fluid has been drawn from the patient. For example, as shown at box 3016, fluid expelled from the body may be collected and a total volume of obtained fluid may be monitored. In that case, the pump can continue to operate until a predetermined fluid volume has been collected from the ureteral and/or bladder catheters. The predetermined fluid volume may be based, for example, on a volume of fluid provided to the patient prior to and during the surgical procedure. As shown at box 3018, application of negative pressure to the ureter and/or kidneys is stopped when the collected total volume of fluid exceeds the predetermined fluid volume.

In other examples, operation of the pump can be determined based on measured physiological parameters of the patient, such as measured creatinine clearance, blood creatinine level, or hematocrit ratio. For example, as shown at box 3020, urine collected form the patient may be analyzed by one or more sensors associated with the catheter and/or pump. The sensor can be a capacitance sensor, analyte sensor, optical sensor, or similar device configured to measure urine analyte concentration. In a similar manner, as shown at box 3022, a patient's blood creatinine or hematocrit level could be analyzed based on information obtain from the patient monitoring sensors discussed hereinabove. For example, a capacitance sensor may be placed in an existing extracorporeal blood system. Information obtained by the capacitance sensor may be analyzed to determine a patient's hematocrit ratio. The measured hematocrit ratio may be compared to certain expected or therapeutically acceptable values. The pump may continue to apply negative pressure to the patient's ureter and/or kidney until measured values within the therapeutically acceptable range are obtained. Once a therapeutically acceptable value is obtained, application of negative pressure may be stopped as shown at box 3018.

In other examples, patient body weight may be measured to assess whether fluid is being removed from the patient by the applied negative pressure therapy. For example, a patient's measured bodyweight (including fluid introduced during a fluid resuscitation procedure) can be compared to a patient's dry body weight. As used herein, dry weights is defined as normal body weight measured when a patient is not over-diluted. For example, a patient who is not experiencing one or more of: elevated blood pressure, lightheadedness or cramping, swelling of legs, feet, arms, hands, or around the eyes, and who is breathing comfortably, likely does not have excess fluid. A weight measured when the patient is not experiencing such symptoms can be a dry body weight. Patient weight can be measured periodically until the measured weight approaches the dry body weight. When the measured weight approaches (e.g., is within between 5% and 10% of dry body weight), as shown at box 3018, application of negative pressure can be stopped.

### EXPERIMENTAL EXAMPLES

Inducement of negative pressure within the renal pelvis of farm swine was performed for the purpose of evaluating effects of negative pressure therapy on renal congestion in the kidney. An objective of these studies was to demonstrate whether a negative pressure delivered into the renal pelvis significantly increases urine output in a swine model of renal congestion. In Example 1, a pediatric Fogarty catheter, normally used in embolectomy or bronchoscopy applications, was used in the swine model solely for proof of principle for inducement of negative pressure in the renal pelvis. It is not suggested that a Fogarty catheter be used in humans in clinical settings to avoid injury of urinary tract tissues.

### Example 1

Method

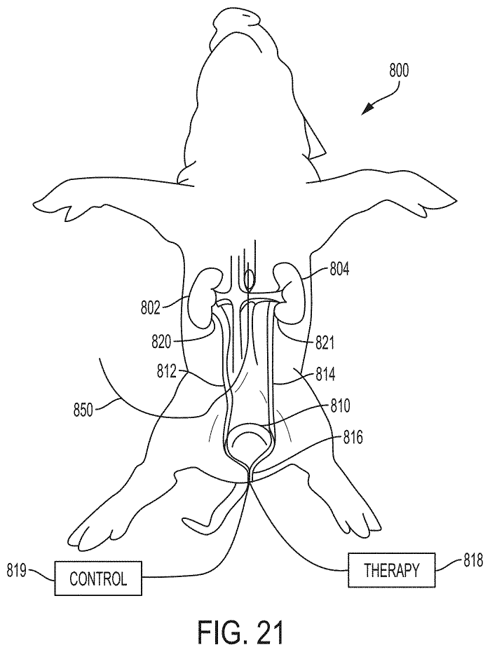

Four farm swine 800 were used for purposes of evaluating effects of negative pressure therapy on renal congestion in the kidney. As shown in FIG. 21, pediatric Fogarty catheters 812, 814 were inserted to the renal pelvis region 820, 821 of each kidney 802, 804 of the four swine 800. The catheters 812, 814 were deployed within the renal pelvis region by inflating an expandable balloon to a size sufficient to seal the renal pelvis and to maintain the position of the balloon within the renal pelvis. The catheters 812, 814 extend from the renal pelvis 802, 804, through a bladder 810 and urethra 816, and to fluid collection containers external to the swine.

Urine output of two animals was collected for a 15 minute period to establish a baseline for urine output volume and rate. Urine output of the right kidney 802 and the left kidney 804 were measured individually and found to vary considerably. Creatinine clearance values were also determined.

Renal congestion (e.g., congestion or reduced blood flow in the veins of the kidney) was induced in the right kidney 802 and the left kidney 804 of the animal 800 by partially occluding the inferior vena cava (IVC) with an inflatable balloon catheter 850 just above to the renal vein outflow. Pressure sensors were used to measure IVC pressure. Normal IVC pressures were 1-4 mmHg. By inflating the balloon of the catheter 850 to approximately three quarters of the IVC diameter, the IVC pressures were elevated to between 15-25 mmHg. Inflation of the balloon to approximately three quarters of IVC diameter resulted in a 50-85% reduction in urine output. Full occlusion generated IVC pressures above 28 mmHg and was associated with at least a 95% reduction in urine output.

One kidney of each animal 800 was not treated and served as a control (“the control kidney 802”). The ureteral catheter 812 extending from the control kidney was connected to a fluid collection container 819 for determining fluid levels. One kidney (“the treated kidney 804”) of each animal was treated with negative pressure from a negative pressure source (e.g., a therapy pump 818 in combination with a regulator designed to more accurately control the low magnitude of negative pressures) connected to the ureteral catheter 814. The pump 818 was an Air Cadet Vacuum Pump from Cole-Parmer Instrument Company (Model No. EW-07530-85). The pump 818 was connected in series to the regulator. The regulator was an V-800 Series Miniature Precision Vacuum Regulator—1/8 NPT Ports (Model No. V-800-10-W/K), manufactured by Airtrol Components Inc.

The pump 818 was actuated to induce negative pressure within the renal pelvis 820, 821 of the treated kidney according to the following protocol. First, the effect of negative pressure was investigated in the normal state (e.g., without inflating the IVC balloon). Four different pressure levels (−2, −10, −15, and −20 mmHg) were applied for 15 minutes each and the rate of urine produced and creatinine clearance were determined. Pressure levels were controlled and determined at the regulator. Following the −20 mmHg therapy, the IVC balloon was inflated to increase the pressure by 15-20 mmHg. The same four negative pressure levels were applied. Urine output rate and creatinine clearance rate for the congested control kidney 802 and treated kidney 804 were obtained. The animals 800 were subject to congestion by partial occlusion of the IVC for 90 minutes. Treatment was provided for 60 minutes of the 90 minute congestion period.

Following collection of urine output and creatinine clearance data, kidneys from one animal were subjected to gross examination then fixed in a 10% neutral buffered formalin. Following gross examination, histological sections were obtained, examined, and magnified images of the sections were captured. The sections were examined using an upright Olympus BX41 light microscope and images were captured using an Olympus DP25 digital camera. Specifically, photomicrograph images of the sampled tissues were obtained at low magnification (20× original magnification) and high magnification (100× original magnification). The obtained images were subjected to histological evaluation. The purpose of the evaluation was to examine the tissue histologically and to qualitatively characterize congestion and tubular degeneration for the obtained samples.

Surface mapping analysis was also performed on obtained slides of the kidney tissue. Specifically, the samples were stained and analyzed to evaluate differences in size of tubules for treated and untreated kidneys. Image processing techniques calculated a number and/or relative percentage of pixels with different coloration in the stained images. Calculated measurement data was used to determine volumes of different anatomical structures.

### Results

Urine Output and Creatinine Clearance

Urine output rates were highly variable. Three sources of variation in urine output rate were observed during the study. The inter-individual and hemodynamic variability were anticipated sources of variability known in the art. A third source of variation in urine output, upon information and belief believed to be previously unknown, was identified in the experiments discussed herein, namely, contralateral intra-individual variability in urine output.

Baseline urine output rates were 0.79 ml/min for one kidney and 1.07 ml/min for the other kidney (e.g., a 26% difference). The urine output rate is a mean rate calculated from urine output rates for each animal.

When congestion was provided by inflating the IVC balloon, the treated kidney urine output dropped from 0.79 ml/min to 0.12 ml/min (15.2% of baseline). In comparison, the control kidney urine output rate during congestion dropped from 1.07 ml/min to 0.09 ml/min (8.4% of baseline). Based on urine output rates, a relative increase in treated kidney urine output compared to control kidney urine output was calculated, according to the following equation:

(Therapy Treated/Baseline Treated)/(Therapy Control /Baseline Control)=Relative increase (0.12 ml/min/0.79 ml/min)/(0.09 ml/min/1.07 ml/min)=180.6%

Thus, the relative increase in treated kidney urine output rate was 180.6% compared to control. This result shows a greater magnitude of decrease in urine production caused by congestion on the control side when compared to the treatment side. Presenting results as a relative percentage difference in urine output adjusts for differences in urine output between kidneys.

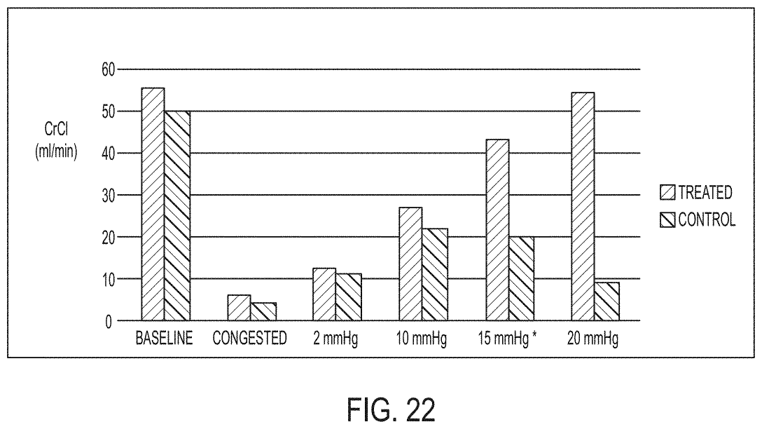

Creatinine clearance measurements for baseline, congested, and treated portions for one of the animals are shown in FIG. 22.

Gross Examination and Histological Evaluation

Based on gross examination of the control kidney (right kidney) and treated kidney (left kidney), it was determined that the control kidney had a uniformly dark red-brown color, which corresponds with more congestion in the control kidney compared to the treated kidney. Qualitative evaluation of the magnified section images also noted increased congestion in the control kidney compared to the treated kidney. Specifically, as shown in Table 1, the treated kidney exhibited lower levels of congestion and tubular degeneration compared to the control kidney. The following qualitative scale was used for evaluation of the obtained slides.

### Congestion

| Lesion | Score |
|---|---|
| None: | 0 |
| Mild: | 1 |
| Moderate: | 2 |
| Marked: | 3 |
| Severe: | 4 |

### Tubular Degeneration

| Lesion | Score |
|---|---|
| None: | 0 |
| Mild: | 1 |
| Moderate: | 2 |
| Marked: | 3 |
| Severe: | 4 |

TABLE 1
TABULATED RESULTS

| | | Histologic lesions | | |
|---|---|---|---|---|
| | | | Tubular | |
| Animal ID/Organ/ | Slide | | hyaline | |
| Gross lesion | number | Congestion | casts | Granulomas |
| 6343/Left Kidney/ | R16-513-1 | 1 | 1 | 0 |
| Normal | | | | |
| 6343/Left Kidney/ | R16-513-2 | 1 | 1 | 0 |
| Normal with | | | | |
| hemorrhagic streak | | | | |
| 6343/Right Kidney/ | R16-513-3 | 2 | 2 | 1 |
| Congestion | | | | |
| 6343/Right Kidney/ | R16-513-4 | 2 | 1 | 1 |
| Congestion | | | | |

As shown in Table 1, the treated kidney (left kidney) exhibited only mild congestion and tubular degeneration. In contrast, the control kidney (right kidney) exhibited moderate congestion and tubular degeneration. These results were obtained by analysis of the slides discussed below.

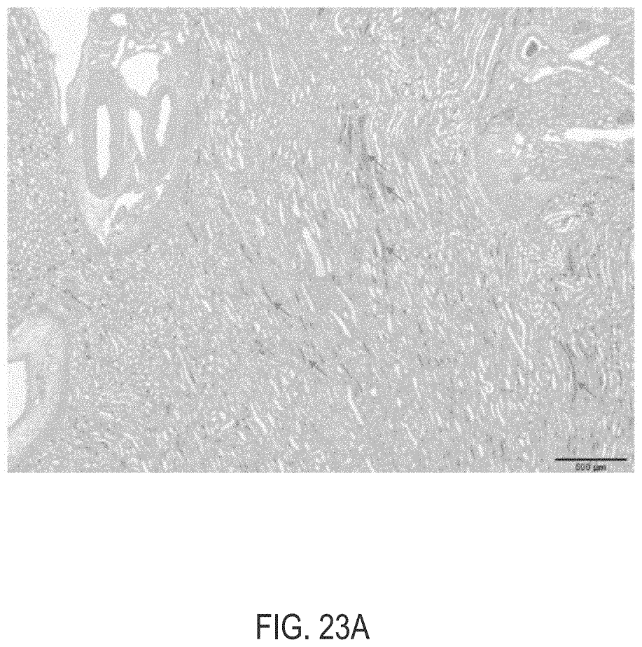

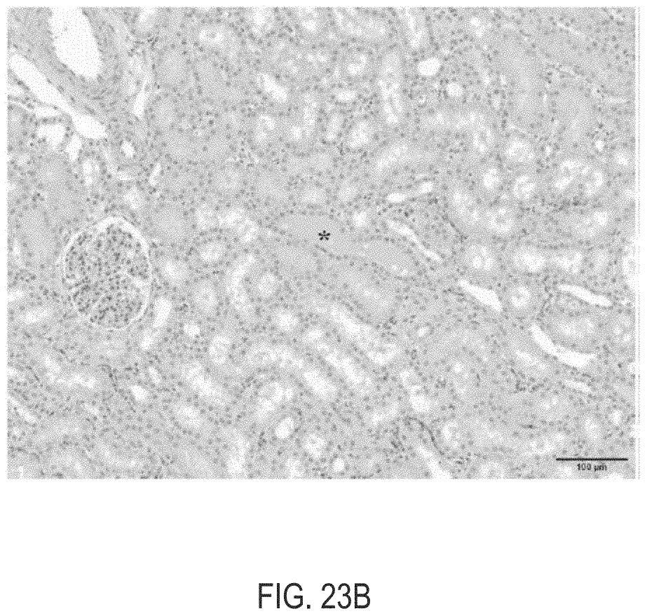

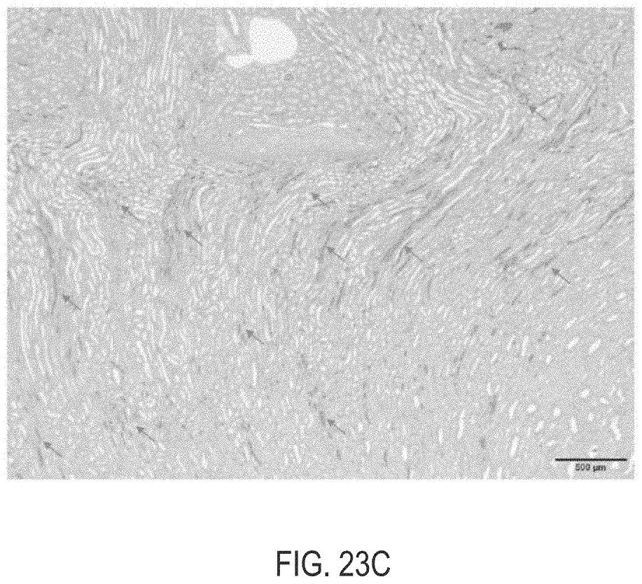

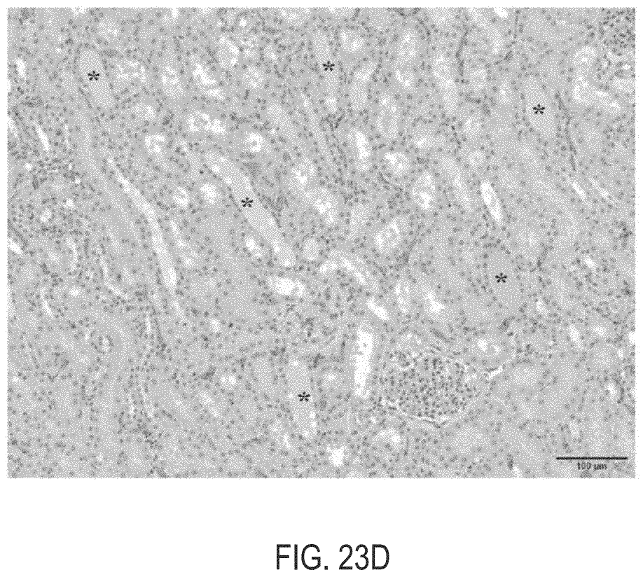

FIGS. 23A and 23B are low and high magnification photomicrographs of the left kidney (treated with negative pressure) of the animal. Based on the histological review, mild congestion in the blood vessels at the corticomedullary junction was identified, as indicated by the arrows. As shown in FIG. 23B, a single tubule with a hyaline cast (as identified by the asterisk) was identified.

FIGS. 23C and 23D are low and high resolution photomicrographs of the control kidney (right kidney). Based on the histological review, moderate congestion in the blood vessel at the corticomedullary junction was identified, as shown by the arrows in FIG. 23C. As shown in FIG. 23D, several tubules with hyaline casts were present in the tissue sample (as identified by asterisks in the image). Presence of a substantial number of hyaline casts is evidence of hypoxia.

Surface mapping analysis provided the following results. The treated kidney was determined to have 1.5 times greater fluid volume in Bowman's space and 2 times greater fluid volume in tubule lumen. Increased fluid volume in Bowman's space and the tubule lumen corresponds to increased urine output. In addition, the treated kidney was determined to have 5 times less blood volume in capillaries compared to the control kidney. The increased volume in the treated kidney appears to be a result of (1) a decrease in individual capillary size compared to the control and (2) an increase in the number of capillaries without visible red blood cells in the treated kidney compared to the control kidney, an indicator of less congestion in the treated organ.

Summary

These results indicate that the control kidney had more congestion and more tubules with intraluminal hyaline casts, which represent protein-rich intraluminal material, compared to the treated kidney. Accordingly, the treated kidney exhibits a lower degree of loss of renal function. While not intending to be bound by theory, it is believed that as severe congestion develops in the kidney, hypoxemia of the organ follows. Hypoxemia interferes with oxidative phosphorylation within the organ (e.g., ATP production). Loss of ATP and/or a decrease in ATP production inhibits the active transport of proteins causing intraluminal protein content to increase, which manifests as hyaline casts. The number of renal tubules with intraluminal hyaline casts correlates with the degree of loss of renal function. Accordingly, the reduced number of tubules in the treated left kidney is believed to be physiologically significant. While not intending to be bound by theory, it is believed that these results show that damage to the kidney can be prevented or inhibited by applying negative pressure to a catheter inserted into the renal pelvis to facilitate urine output.

### Example 2

Method

Inducement of negative pressure within the renal pelvis of farm swine was performed for the purpose of evaluating effects of negative pressure therapy on hemodilution of the blood. An objective of these studies was to demonstrate whether a negative pressure delivered into the renal pelvis significantly increases urine output in a swine model of fluid resuscitation.

Two pigs were sedated and anesthetized using ketamine, midazolam, isoflurane and propofol. One animal (#6543) was treated with a ureteral catheter and negative pressure therapy as described herein. The other, which received a Foley type bladder catheter, served as a control (#6566). Following placement of the catheters, the animals were transferred to a sling and monitored for 24 hours.

Fluid overload was induced in both animals with a constant infusion of saline (125 mL/hour) during the 24 hour follow-up. Urine output volume was measured at 15 minute increments for 24 hours. Blood and urine samples were collected at 4 hour increments. As shown in FIG. 20, a therapy pump 818 was set to induce negative pressure within the renal pelvis 820, 821 (shown in FIG. 20) of both kidneys using a pressure of −45 mmHg (+/−2 mmHg).

Results

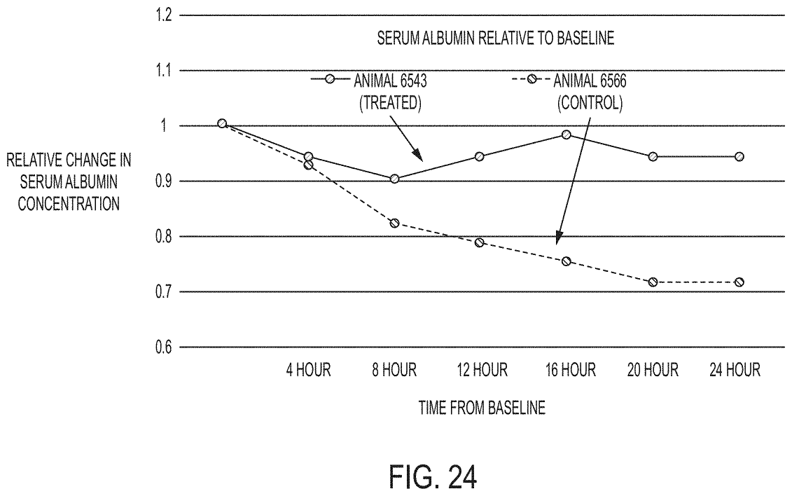

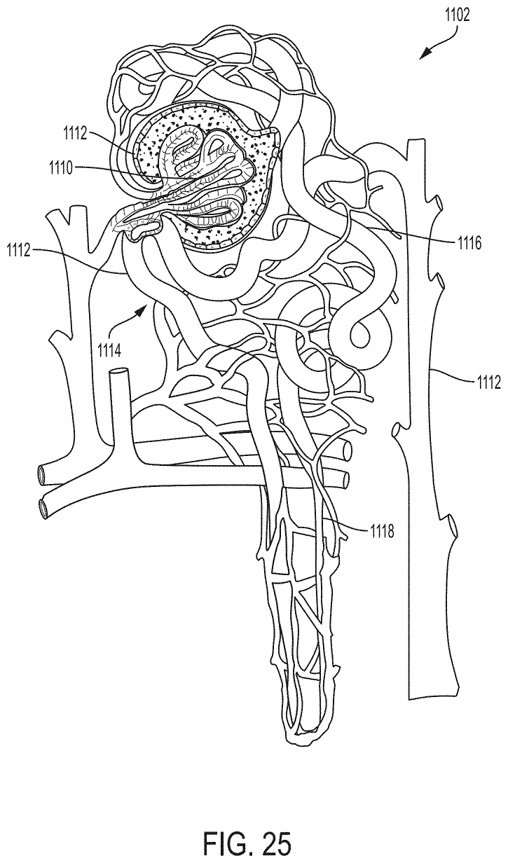

Both animals received 7 L of saline over the 24 hour period. The treated animal produced 4.22 L of urine while the control produced 2.11 L. At the end of 24 hours, the control had retained 4.94 L of the 7 L administered, while the treated animal retained 2.81 L of the 7 L administered. FIG. 24 illustrates the change in serum albumin. The treated animal had a 6% drop in the serum albumin concentration over 24 hours, while the control animal had a 29% drop.

Summary

While not intending to be bound by theory, it is believed that the collected data supports the hypothesis that fluid overload induces clinically significant impact on renal function and, consequently induces hemodilution. In particular, it was observed that administration of large quantities of intravenous saline cannot be effectively removed by even healthy kidneys. The resulting fluid accumulation leads to hemodilution. The data also appears to support the hypothesis that applying negative pressure diuresis therapy to fluid overloaded animals can increase urine output, improve net fluid balance and decrease the impact of fluid resuscitation on development of hemodilution.

The preceding examples and embodiments of the invention have been described with reference to various examples. Modifications and alterations will occur to others upon reading and understanding the foregoing examples. Accordingly, the foregoing examples are not to be construed as limiting the disclosure.

What is claimed is:

1. A fluid collection catheter configured to be deployed in a bladder of a patient, comprising:
an elongated tube comprising a proximal portion configured for placement in a urethra of the patient, a distal portion comprising a distal end, and a sidewall extending between a proximal end and the distal end of the elongated tube defining at least one drainage lumen extending through the tube, the sidewall comprising a drainage portion which allows fluid to pass through the sidewall and into the drainage lumen; and
a tissue support comprising at least a first flange comprising a central portion connected to the distal portion of the elongated tube and an outer portion extending radially and axially therefrom, the first flange being configured to be deployed in the bladder to maintain the distal end of the elongated tube at a predetermined position in the bladder,
wherein, when deployed, the tissue support defines a three-dimensional shape of sufficient size to permit flow of at least a portion of fluid contained in the bladder from the bladder through the drainage portion of the elongated tube to the at least one drainage lumen.

2. The catheter of claim 1, wherein the at least one flange is configured to transition from a retracted position in which at least a portion of a proximally facing surface of the first flange contacts an outer surface of the sidewall of the elongated tube, to a deployed position in which the portion of the proximally facing surface of the first flange is spaced apart from the sidewall.

3. The catheter of claim 1, wherein, when deployed in the patient's bladder, the tissue support is configured to maintain a volume of the three dimensional shape when an interior of the bladder is exposed to an internal negative pressure.

4. The catheter of claim 1, wherein, when deployed in the patient's bladder, the tissue support is configured to maintain a volume of the three dimensional shape when an interior of the bladder is exposed to an internal negative pressure of from about 0.1 mmHg to about 150 mmHg.

5. The catheter of claim 1, wherein, when deployed in the patient's bladder, the tissue support is configured to inhibit any portion of the bladder wall from occluding or obstructing ureteral orifices of the bladder upon delivery of negative pressure to the bladder through the drainage lumen of the tube.

6. The catheter of claim 1, wherein the first flange has a maximum outer diameter of from about 10 mm to about 100 mm.

7. The catheter of claim 1, wherein a height of the first flange is from about 10 mm to about 100 mm.

8. The catheter of claim 1, wherein the elongated tube has an outer diameter of from about 0.5 mm to about 10 mm.

9. The catheter of claim 1, wherein the elongated tube has an inner diameter of from about 0.5 mm to about 9 mm.

10. The catheter of claim 1, wherein, when deployed in the patient's bladder, the three dimensional shape has a volume of from 0.1 cm³ to 500 cm³.

11. The catheter of claim 1, wherein the drainage portion of the sidewall comprises a perforated section of tubing comprising at least one perforation permitting fluid to flow through the sidewall of the elongated tube into the at least one drainage lumen.

12. The catheter of claim 11, wherein the at least one perforation has one or more shapes, each shape being selected from at least one of a circular shape, an elliptical shape, a square shape, a regular polygonal shape, an irregular circular shape, an irregular polygonal shape, or combinations thereof.

13. The catheter of claim 11, wherein the at least one perforation has a diameter of about 0.05 mm to about 2.0 mm.

14. The catheter of claim 11, wherein, when deployed in the patient's bladder, the tissue support is configured to inhibit any portion of a wall of the bladder from occluding the at least one perforation of the drainage portion upon delivery of negative pressure to an interior of the bladder through the drainage lumen of the elongated tube.

15. The catheter of claim 1, wherein, when deployed in the patient's bladder, the first flange comprises a distally facing dome-shaped surface extending radially outwardly and proximally from the sidewall of the elongated tube.

16. The catheter of claim 15, wherein the first flange comprises at least one radial slit extending from an outer edge of the flange radially inwardly toward the central portion of the flange.

17. The catheter of claim 16, wherein the first flange comprises a plurality of radial slits which at least partially separate petal portions of the flange, and wherein a distance between adjacent petal portions increases as the flange transitions to the deployed position.

18. The catheter of claim 1, wherein the first flange comprises at least one perforation extending between a proximal surface and a distal surface of the flange, and wherein the at least one perforation is positioned to permit negative pressure to pass through the flange to other portions of the bladder.

19. The catheter of claim 1, wherein the at least one first flange comprises a medical grade elastomeric polymer material.

20. The catheter of claim 19, wherein the elastomeric polymer material comprises one or more of silicone, thermoplastic polyurethane, or a composites of a silicone or a polyurethane and a metallic component.

21. The catheter of claim 1, wherein the at least one first flange comprises silicone having a shore hardness of between about Shore 20 A and Shore 100 A.

22. The catheter of claim 1, wherein the central portion of the at least one first flange comprises a collar slidably connected to the sidewall of the elongated tube configured to slide along the sidewall of the tube to adjust a position of the at least one first flange.

23. The catheter of claim 1, further comprising at least one second flange comprising a central opening connected to the sidewall of the elongated tube at a position proximal to the at least one first flange.

24. The catheter of claim 23, wherein, upon application of pressure to a proximally facing surface of the second flange, the second flange transitions to a concave configuration.

25. The catheter of claim 23, wherein, when deployed in the patient's bladder, a proximally facing surface of the second flange is configured to contact a portion of an inferior portion of the bladder wall surrounding a urethra opening into the bladder.

26. The catheter of claim 23, wherein an outer diameter of the second flange is greater than an outer diameter of the first flange.

27. The catheter of claim 23, wherein the distal portion of the elongated tube comprises a telescoping tube comprising an inner tube slidably received in an outer tube for adjusting a distance between the first flange and the second flange.

28. The catheter of claim 27, wherein the first flange is connected to the inner tube and the second flange is connected to the outer tube.

29. The catheter of claim 23, further comprising at least one third flange comprising a central portion connected to the distal end of the elongated tube and an outer portion extending therefrom, the third flange being configured to support a superior portion of the bladder wall.

30. The catheter of claim 29, wherein an outer diameter of the third flange is less than an outer diameter of the first flange and the second flange.

31. The catheter of claim 1, further comprising a delivery catheter comprising a proximal end configured to remain external to the body, a distal end for insertion into the bladder, a sidewall extending therebetween, and at least one lumen sized to receive the elongated tube and tissue support, wherein the delivery catheter is configured to maintain the tissue support in a retracted position, in which at least a portion of a lower surface of the flange contacts the sidewall of the elongated tube, during insertion of the tissue support to the bladder of the patient.

32. The catheter of claim 31, wherein the delivery catheter has an inner diameter of from about 1.0 mm to about 20 mm.

33. The catheter of claim 31, wherein the at least one flange is biased to a deployed position, such that when pushed from the distal end of the delivery catheter, the at least one flange adopts its deployed configuration.

34. A method of inducing a negative pressure to a urinary tract of a patient for enhancing urine excretion therefrom, the method comprising:
inserting a distal portion of an elongated tube of a urine collection catheter into the urinary tract, the elongated tube comprising a proximal portion configured for placement in a urethra of the patient, a distal portion comprising a distal end, and a sidewall extending between a proximal end and the distal end of the elongated tube defining at least one drainage lumen extending through the tube, the sidewall comprising a drainage portion which allows fluid to pass through the sidewall and into the drainage lumen;
deploying a tissue support at a predetermined position in the patient's bladder, the tissue support comprising at least one first flange comprising a central portion connected to the distal portion of the elongated tube and an outer portion extending axially and/or radially therefrom, wherein the tissue support is configured to be deployed in the bladder to maintain the distal end of the elongated tube at the predetermined position in the patient's bladder, and wherein, when deployed, the tissue support defines a three-dimensional shape of sufficient size to permit flow of at least a portion of fluid contained in the bladder from the bladder through the drainage portion of the elongated tube to the at least one drainage lumen; and
inducing a negative pressure through the at least one drainage lumen of the elongated tube to draw at least a portion of fluid contained in the bladder from the bladder through the drainage portion of the elongated tube to the at least one drainage lumen.

35. The method of claim 34, wherein, when deployed in the patient's bladder, the tissue support is configured to maintain a volume of the three dimensional shape when an interior of the bladder is exposed to the negative pressure.

36. The method of claim 34, wherein, when deployed in the patient's bladder, the tissue support is configured to maintain a volume of the three dimensional shape when an interior of the bladder is exposed to an internal negative pressure of from about 0.1 mmHg to about 150 mmHg.

37. The method of claim 34, wherein inducing the negative pressure in the drainage lumen comprises coupling a mechanical pump to the proximal end of the drainage lumen to draw urine from the bladder into the drainage lumen through the drainage portion of the sidewall.

38. The method of claim 34, wherein inducing the negative pressure comprises applying a negative pressure of from about 0.1 mmHg to about 150 mmHg to the proximal end of the elongated tube.

39. The method of claim 34, wherein the elongated tube is inserted into the bladder in a delivery catheter, and wherein deploying the tissue support comprises retracting the delivery catheter to expose the at least one flange.

40. The method of claim 39, wherein the at least one first flange is biased to adopt a deployed position when the delivery catheter is retracted.

41. The method of claim 34, wherein the tissue support further comprises at least a second flange comprising a central opening connected to the sidewall of the elongated tube at a position proximal to the first flange.

42. The method of claim 41, wherein deploying the tissue support comprises adjusting a position of the second flange such that a proximally facing surface of the second flange contacts a portion of an inferior portion of the bladder wall surrounding a urethra opening into the bladder.

43. The method of claim 41, wherein the tissue support further comprises at least one third flange comprising a central portion connected to the distal end of the elongated tube and an outer portion extending therefrom, and wherein, when negative pressure is applied to the bladder, the third flange supports a superior portion of the bladder wall.

44. A system for drawing urine from a urinary tract of a patient, the system comprising:
a urine collection catheter configured to be deployed in a bladder of a patient comprising:
an elongated tube comprising a proximal portion configured for placement in a urethra of the patient, a distal portion comprising a distal end, and a sidewall extending between a proximal end and the distal end of the elongated tube defining at least one drainage lumen extending through the tube, the sidewall comprising a drainage portion which allows fluid to pass through the sidewall and into the drainage lumen; and
a tissue support comprising at least a first flange comprising a central portion connected to the distal portion of the elongated tube and an outer portion extending radially and/or axially therefrom, the first flange being configured to be deployed in the bladder to maintain the distal end of the elongated tube at a predetermined position in the bladder, wherein, when deployed, the tissue support defines a three-dimensional shape of sufficient size to permit flow of at least a portion of fluid contained in the bladder from the bladder through the drainage portion of the elongated tube to the at least one drainage lumen; and
a pump in fluid connection with the drainage lumen of the elongated tube, wherein the pump is configured to introduce an internal negative pressure through the drainage lumen to the urinary tract of the patient to draw urine from the urinary tract.

45. The system of claim 44, wherein, when deployed in the patient's bladder, the tissue support is configured to maintain a volume of the three dimensional shape when an interior of the bladder is exposed to the internal negative pressure.

46. The system of claim 44, wherein, when deployed in the patient's bladder, the permeable tissue support is configured to maintain a volume of the three dimensional shape when an interior of the bladder is exposed to an internal negative pressure of from about 0.1 mmHg to about 150 mmHg.

47. The system of claim 44, wherein the pump provides a sensitivity of about 10 mmHg or less.

48. The system of claim 44, wherein the pump is configured to provide a negative pressure of from about 0.1 mmHg to about 150 mmHg.

49. The system of claim 44, wherein the pump is configured to provide intermittent negative pressure.

50. The system of claim 44, wherein the pump is configured to alternate between providing negative pressure and providing positive pressure.

51. The system of claim 44, wherein the pump is configured to alternate between providing negative pressure and equalizing pressure to atmosphere.

52. The system of claim 44, wherein the tissue support further comprises at least one second flange comprising a central opening connected to the sidewall of the elongated tube at a position proximal to the first flange.

53. The system of claim 52, wherein, upon application of pressure to a proximally facing surface of the second flange, a surface of the second flange transitions to a concave configuration.

54. The system of claim 52, wherein the tissue support further comprises at least one third flange comprising a central portion connected to the distal end of the elongated tube and an outer portion extending radially and axially therefrom, and wherein, when negative pressure is applied to the bladder, the third flange supports a superior portion of the bladder wall.
